(19) United States
(12) Patent Application Publication
(10) Pub. No.: US 2023/0165272 A1
(43) Pub. Date: Jun. 1, 2023

(54) REFINED ZEIN-ENRICHED AND/OR ZEIN-DEPLETED PROTEIN HYDROLYSATE COMPOSITIONS AND METHODS OF MAKING

(71) Applicant: CARGILL, INCORPORATED, Wayzata, MN (US)

(72) Inventors: Chathurada Sugeeshvarie GAJADEERA, Maple Grove, MN (US); Michael A. PORTER, Maple Grove, MN (US)

(73) Assignee: CARGILL, INCORPORATED, Wayzata, MN (US)

(21) Appl. No.: 17/997,321

(22) PCT Filed: Apr. 29, 2021
(86) PCT No.: PCT/US2021/029886

§ 371 Date: Oct. 27, 2022

Related U.S. Application Data
(60) Provisional application No. 63/018,170, filed on Apr. 30, 2020.

(51) Int. Cl. A23J 3/34; A23J 1/12; A23J 3/14; A23L 33/18; C07K 14/425

(52) U.S. Cl. CPC A23J 3/346; A23J 1/12; A23J 3/14; A23L 33/18; C07K 14/425

(57) ABSTRACT

A method of preparing a refined zein-enriched protein hydrolysate composition and/or a refined zein-depleted protein hydrolysate composition comprising enzymatically hydrolyzing a refined zein-enriched protein composition and/or a refined zein-depleted protein composition that have been separated from a refined, destarched corn gluten meal. The refined zein-enriched protein hydrolysate composition has a protein solubility of from about 15% to about 20% at a pH selected from the group consisting of pH 3.4, pH 7.0, and of both pH 3.4 and pH 7.0. The refined zein-depleted protein hydrolysate composition has a protein solubility of from about 20% to about 35% at a pH selected from the group consisting of pH 3.4, pH 7.0, and of both pH 3.4 and pH 7.0. Alternatively, the refined zein-enriched protein composition and/or the refined zein-depleted protein composition have been separated from a corn protein isolate as described herein. Compositions are also provided.

### TECHNICAL FIELD

The present patent application relates to the field of corn protein. More particularly, the present patent application relates to corn protein fractions.

### BACKGROUND

Corn or maize (Zea mays L.) is a major cereal grain globally and one of the most important food and industrial crops in the US. The main parts of the corn kernel are the endosperm and the germ, which are the prime sources of starch and oil, respectively. Bran and tip cap are the remaining components of the kernel. Different types of proteins are found in the two main constituents: albumins and globulins centralized primarily in the germ, and prolamin type proteins found mostly in the endosperm. The protein content of different corn varieties ranges between 6-12% on a dry basis. About 75% of the total corn protein is located in the endosperm. There are four major classes of protein in corn categorized largely by their solubility in selected solvents. Zein, which is soluble in ethanol, is the prolamin protein in corn (Anderson & Lamsal, 2011). Almost all the zein proteins are present in the endosperm, whereas glutelin, soluble in alkaline solutions is distributed between the endosperm and the germ. The globulins and albumins, soluble in salt and water respectively, are present mainly in the germ and are mainly enzymes and biologically active proteins. The prolamins and glutelins are the major storage proteins that comprise nitrogen for seed development, and constitute 80% of the total nitrogen in the corn kernel.

Zein proteins (α, β, γ and δ-zein) are located in ‘zein-bodies’ distributed in the cytoplasm of corn endosperm cells between starch granules (Duvick, 1961). Zein proteins are insoluble in water but can be solubilized in the presence of alcohol, high concentrations of urea and high concentrations of alkali (pH 11 or above) or anionic detergents. This is mainly attributed to its amino acid composition. Zein is particularly rich in glutamic acid (21-26%), leucine (20%), proline (10%) and alanine (10%), but deficient in basic amino acids. The higher amount of nonpolar amino acids (58.6%) (Shukla & Cheryan, 2001) and the deficiency in basic amino acids result in a protein configuration that is highly hydrophobic contributing to low solubility of zein in water.

### SUMMARY

Corn protein has received little attention from the food industry as a potential food ingredient. Corn protein is not considered to be allergenic, which makes it suitable for widespread use and decreases the cleaning cost associated with mixed allergen/non-allergen production. It has been discovered that corn protein compositions can be recovered from corn gluten meal and prepared by a sequence of destarching, defatting with a water-miscible solvent/water composition having a high water-miscible solvent content, fractionation into a zein-enriched protein fraction composition and a zein-depleted protein fraction composition, and hydrolysis of the zein-enriched protein fraction composition and the zein-depleted protein fraction composition by enzymatic treatments. The resulting protein compositions have unique solubility, organoleptic, and nutritional characteristics that are individually applicable for different food and nutritional supplement applications. In particular, it has been found that the different corn protein compositions prepared by the methods as described herein are physically and chemically different from each other, and additionally have amino acid profiles that are different from each other. These chemical differences and in particular the amino acid profile differences provide unique benefits and facilitate the highest use of each component present in the starting corn gluten meal material.

In an aspect, it has been found that different corn protein compositions prepared by the methods as described herein may be thermally stable, so that thermal treatment of these compositions does not induce protein aggregation or reduced solubility. Thermal stability of the present compositions is particularly beneficial for use in product formats that may be exposed to heat, such as in beverage applications. In an aspect, it has been found that different corn protein compositions prepared by the methods as described herein may be stable under acidic conditions. The protein stability of solubility in acidic conditions is desirable in many foods processes where pH modification occurs.

In an aspect, a method of preparing a refined zein-enriched protein hydrolysate composition and/or a refined zein-depleted protein hydrolysate composition comprises providing a refined zein-enriched protein composition and/or a refined zein-depleted protein composition, wherein the refined zein-enriched protein composition and/or the refined zein-depleted protein composition have been separated from a refined, destarched corn gluten meal.

For purposes of the present disclosure, a refined, destarched corn gluten meal is a destarched corn gluten meal that has been prepared by treatment with a water-miscible solvent/water extraction composition comprising from about 55% to about 99% water-miscible solvent, the refined, destarched corn gluten meal:

i) comprising less than about 2 wt % oil on a dry basis,

ii) having an L* color value of from about 88 to 95, an “a*” color value ranging from about −0.5 to 1.5, and a “b*” color value ranging from about 10 to 25,

iii) having a soluble carbohydrate concentration of 40 g/kg or less, and

iv) comprising at least about 85 wt % protein on a dry basis.

An enzyme is added to the refined zein-enriched protein composition to provide a zein-enriched protein suspension, and/or an enzyme is added to the refined zein-depleted protein composition to provide a zein-depleted protein suspension.

The pH and temperature of the zein-enriched protein suspension and/or the zein-depleted protein suspension is controlled to hydrolyze protein in the zein-enriched protein suspension and/or the zein-depleted protein suspension and the hydrolysis of the zein-enriched protein suspension and/or the zein-depleted protein suspension is terminated to provide
i) a refined zein-enriched protein hydrolysate composition having a protein solubility of from about 15% to about 20% at a pH selected from the group consisting of pH 3.4, pH 7.0, and of both pH 3.4 and pH 7.0; and/or
ii) a refined zein-depleted protein hydrolysate composition having a protein solubility of from about 20% to about 35% at a pH selected from the group consisting of pH 3.4, pH 7.0, and of both pH 3.4 and pH 7.0.

In an aspect, the refined zein-enriched protein composition and/or the refined zein-depleted protein composition have been separated from a corn protein isolate, the corn protein isolate having a corn protein concentration of at least 85% (ds), and wherein from about 40% to about 70% of the corn protein in the corn protein isolate is soluble in a 65% ethanol/water solution at a temperature of 60° C.

In an aspect, the method further comprises a step of separating the water-soluble proteins from the water-insoluble proteins in the refined zein-enriched protein hydrolysate composition to provide a water-soluble refined zein-enriched protein hydrolysate composition and a water-insoluble refined zein-enriched protein hydrolysate composition.

In an aspect, the method further comprises a step of separating the water-soluble proteins from the water-insoluble proteins in the refined zein-depleted protein hydrolysate composition to provide a water-soluble refined zein-depleted protein hydrolysate composition and a water-insoluble refined zein-depleted protein hydrolysate composition.

In an aspect, refined zein-enriched protein hydrolysate compositions prepared by any of the methods described herein are provided.

In an aspect, the method as described herein advantageously can provide two different protein composition products, one of which is a refined zein-enriched protein hydrolysate composition having enhanced water-solubility as compared to a like zein-enriched protein composition that has not been hydrolyzed. The other protein composition is a refined zein-depleted protein hydrolysate composition having enhanced water-solubility as compared to a like zein-depleted protein composition that has not been hydrolyzed.

In an aspect, the process as described herein advantageously can provide four additional protein composition products obtained from refined zein-enriched protein hydrolysate composition and the refined zein-depleted protein hydrolysate composition, two of which are corn protein compositions being soluble in water, and two of which are corn protein compositions being insoluble in water. One of the two corn protein compositions that is soluble in water is a water-soluble refined zein-enriched protein hydrolysate composition, and the other is a water-soluble refined zein-depleted protein hydrolysate composition. One of the two corn protein compositions that are insoluble in water is an insoluble refined zein-enriched protein hydrolysate composition, and the other is an insoluble refined zein-depleted protein hydrolysate composition. As described above, each of these four resulting zein-enriched or zein-depleted protein compositions have unique physical, chemical and nutritional properties with respect to each other, and therefore have different optimal uses.

More particularly, in an aspect the present process provides a choice of two different corn protein hydrolysate compositions having improved solubility in water for use in various food and beverage applications and in nutritional supplement applications. Because one of these soluble corn protein compositions is obtained from a zein-enriched protein composition and the other is obtained from a zein-depleted protein composition, the respective soluble corn protein compositions will exhibit different physical properties and will have different amino acid profiles. The soluble corn protein hydrolysate compositions obtained from a zein-enriched protein composition and from a zein-depleted protein composition will therefore provide nutritional benefits that are different from each other, and additionally will have different optimal product uses. Additionally, it will be recognized that solubility is not the only possible functional consequence of hydrolysis. Thus, even if the solubility of a given composition is not increased by hydrolysis, additional benefits may arise from the modification obtained by the hydrolysis step.

Likewise, in an aspect the present process provides a choice of two different corn protein hydrolysate compositions having reduced solubility in water for use in various non-liquid foods, such as extrusion products (for example, extruded snack products), meat replacement products and nutritional supplement applications. Because one of these reduced solubility corn protein hydrolysate compositions is obtained from a zein-enriched protein composition and the other is obtained from a zein-depleted protein composition, the respective reduced solubility corn protein hydrolysate compositions will exhibit different physical properties and will have different amino acid profiles. The reduced solubility corn protein hydrolysate compositions obtained from a zein-enriched protein composition and from a zein-depleted protein composition will therefore provide nutritional benefits that are different from each other, and additionally will have different optimal product uses.

In an aspect, the present process provides a product that may be used as a supplementary amino acid or alternative protein source to provide desired amino acids that may otherwise be missing in a diet as defined by the Protein Digestibility Corrected Amino Acid Score (PDCAAS) method. In an aspect, the present process provides a product that may be used for blending with foods having a different amino acid profile to provide desired amino acids that may otherwise be missing in a food product or a diet as defined by the Protein Digestibility Corrected Amino Acid Score (PDCAAS) method. Because the amino acid profile of each of the different protein composition products is different from the others, the different protein products as described herein may advantageously be used in unique blends with supplements or other food components to provide a desired amino acid profile.

Moreover, it has been discovered that by selection of the corn protein starting material and controlling the degree of hydrolysis of the proteins, the resulting corn protein hydrolysates in an aspect may exhibit excellent flavor characteristics. In an aspect, the resulting corn protein hydrolysates may exhibit a low degree of perceptible bitterness flavor as evaluated by test panel analysis.

The enhanced solubility of the corn protein hydrolysate compositions described herein can facilitate use of the corn protein hydrolysate in multiple product categories, such as food and beverages. In an aspect, corn protein hydrolysate can be included as a protein source additive in a variety of non-liquid food products. The present corn protein hydrolysate compositions advantageously can enhance the protein content of the food products without introducing objectionable flavors. Additionally, the corn protein hydrolysate described herein can exhibit superior organoleptic properties in food and beverage products. Enhanced solubility can increase other functionalities important in food, and protein modification can change those functionalities with no change in solubility.

Corn protein is a valuable source of protein for nutrition. Nutritional benefit can be described in many ways and protein consumption has well-described effects on physiology. Leucine is one of the amino acids found in corn protein, and corn protein is one of the richer sources of leucine among proteins. Leucine is especially important for stimulation of muscle protein synthesis. This is of interest to consumers at all ages, but especially among the elderly. Younger consumers, interested in increasing their muscle mass, often consume proteins containing ample leucine. Some of these proteins are expensive or only available from animal sources. Corn protein is less expensive than most animal proteins and has the sustainability benefits of being plant based. One of the common ways that people seeking a muscle protein synthesis benefit consume protein is as a beverage. Unmodified corn protein has poor solubility and dispersibility in water, but the product of the current invention may be better suited to making a beverage. With improved solubility and low bitterness, the corn protein can be formulated alone or in combination with other proteins to create a nutritious beverage with desirable sensory properties. The modified protein may be more suitable for use in other applications as well.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a process flow diagram illustrating an aspect of the process as described herein.

FIG. 2 is a process flow diagram illustrating an aspect of the process as described herein.

FIG. 3 is a graph showing solubility of protein compositions as described herein at pH 3.4 and pH 7.0.

FIG. 4 is a graph showing solubility of protein compositions as described herein with and without thermal treatment.

### DETAILED DESCRIPTION

In the present disclosure, “Protein solubility” refers to the concentration of the protein that is present in the liquid phase relative to the amount of protein that is present in the liquid and solid phase at equilibrium. Protein solubility can be reported as a percentage and is determined by measuring protein content in the supernatant after applying centrifugal force to a solution prepared at specific protein content, pH and salt concentration, relative to the total protein in the solution prior to centrifugation.

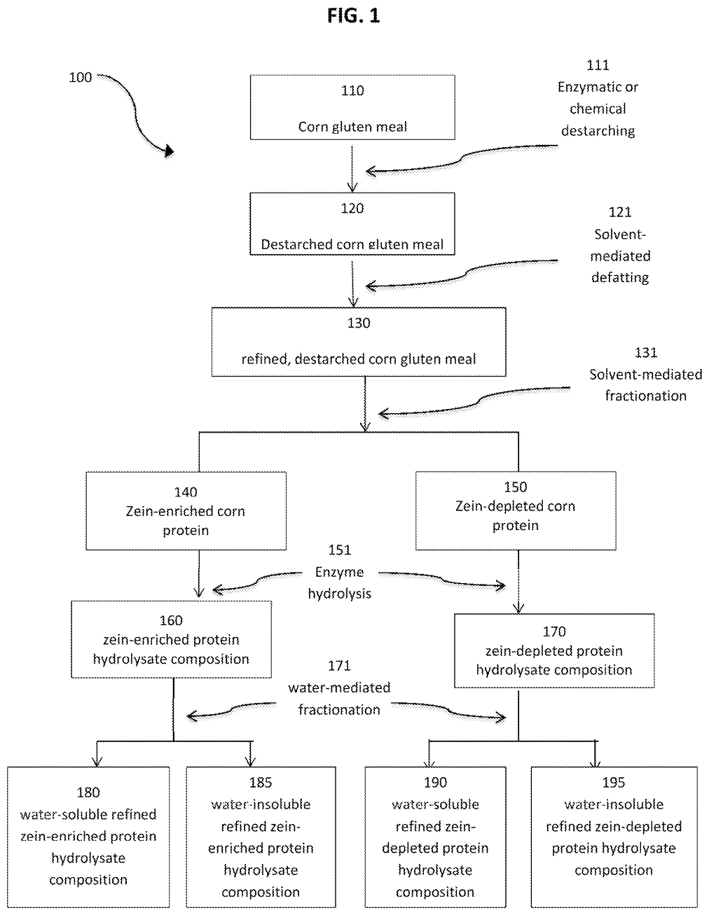

FIG. 1 shows an exemplary process 100 for preparing a refined zein-enriched protein hydrolysate composition and/or a refined zein-depleted protein hydrolysate composition, and optionally further preparing water-soluble and/or water-insoluble compositions from the refined zein-enriched protein hydrolysate composition and/or the refined zein-depleted protein hydrolysate compositions.

As shown, process 100 includes providing corn gluten meal (“CGM”) 110 as a first staring material. CGM is obtained from corn, typically by wet milling to separate corn kernels into products such as starch, protein, fiber and oil. In an aspect, the corn gluten meal comprises at least about 65 weight percent (wt %), at least about 70 wt %, or at least about 80 wt % protein on a dry basis (db). In aspects, the concentration of protein may range from about 65 to 80 wt % (db), or about 70 to 80 wt % (db), or about 75 to 80 wt % (db). The concentration of protein throughout this present disclosure is determined by nitrogen concentration as provided in accordance with AACCI 46-30.01 (Crude Protein-Combustion Method) using a nitrogen analyzer (LECO TruSpecN™, St. Joseph, Mich. USA) and a conversion factor of 6.25.

The CGM is destarched by conventional enzymatic or chemical destarching methods 111, such as by enzyme or chemical hydrolysis and a subsequent separation step to hydrolyze and remove, respectively, the majority of starch components contained in the CGM to provide destarched corn gluten meal 120.

In an aspect, the destarched corn gluten meal is prepared by contacting the CGM composition with carbohydrate hydrolyzing enzymes (carbohydrases) that break-down the starch and/or non-starch complex carbohydrates, such as fiber, into water-soluble carbohydrates. The resulting protein composition is then separated from the aqueous stream. This protein composition may optionally be further filtered to remove low protein content components. Optionally, a washing step can be used during or after filtration to increase protein content of the resulting protein concentrate.

In an aspect, the destarched gluten meal has a residual insoluble starch solids in the range from about 0.1 wt % to 3.0 wt % (ds), as measured by Ewers polarimetric method ISO 10520:1997. In at least certain aspects, the residual starch solids in the destarched corn gluten meal are in the range from about 0.1 to 2.0 wt % (ds), or about 0.1 to 1.0 wt % (ds), or about 0.1 to 0.75 wt % (ds). Methods of measurement are described, for example, in WO2016/154441 and WO 2018/058150 assigned to Cargill Incorporated, which are incorporated herein by reference.

In an aspect, the destarched corn gluten meal has a protein concentration of at least about 65% (dry solids). In an aspect, the destarched corn gluten meal has a protein concentration of at least about 70% (dry solids). In an aspect, the destarched corn gluten meal has a protein concentration of at least about 75% (dry solids). In an aspect, the destarched corn gluten meal has a protein concentration of at least about 80% (dry solids).

In an aspect, the destarched corn gluten meal is the corn protein concentrate described in U.S. Pat. No. 9,226,515 (assigned to Cargill Incorporated, and incorporated herein by reference). A typical analysis of such corn protein concentrate (e.g., Empyreal® 75, Cargill, Incorporated, Wayzata, Minn.) comprises about 75 wt % to 80 wt % protein on a dry weight basis, about 4.5% fat, about 5% soluble carbohydrates, and other nutrients (as-is basis), and has a bright yellow or gold color.

In an aspect, the protein concentration of the destarched corn gluten meal can be further increased by defatting the protein containing material. Defatting can be accomplished using any method known in the art, for instance by using one or more solvents and/or degrading the fats with enzymes. Examples of solvents that can be used include hexane, isohexane, alcohols and mixtures thereof. Examples of enzymes that can be used include lipases and the like. The fats can subsequently be separated from the protein concentrate using any method known in the art, for example filtration, floatation, and/or centrifugation.

In an aspect, the destarched corn gluten meal is additionally treated with an oxidant, to provide corn protein composition starting material having a free sulfite concentration of less than 150 ppm on an as-is basis. In an aspect, the oxidant can be, for example but not limited to, hydrogen peroxide, ozone gas, air, sodium hypochlorite, a combination of potassium bromate and ethanol, catalase, peroxidase, or a combination thereof. In an aspect, the oxidant is hydrogen peroxide. Methods for such treatment are described in WO 2017/165748, assigned to Cargill Incorporated, which is incorporated herein by reference.

The destarched corn gluten meal is then further treated in a solvent-mediated defatting step 121, by application of a water-miscible solvent/water defatting composition comprising from about 55% to about 90% water-miscible solvent to provide refined, destarched corn gluten meal 130. In an aspect, the water-miscible solvent is ethanol. In an aspect, the water-miscible solvent is isopropanol or other water-miscible solvent, and mixtures thereof.

The solvent-mediated defatting step is carried out for a time and under conditions such that the resulting refined, destarched corn gluten meal i) comprises less than about 2 wt % oil on a dry basis; ii) has an L* color value of from about 88 to 95, an “a*” color value ranging from about −0.5 to 1.5, and a “b*” color value ranging from about 10 to 25; iii) has a soluble carbohydrate concentration of 40 g/kg or less; and iv) comprises at least about 85 wt % protein on a dry basis.

It has been found that defatting of the destarched corn gluten meal with the water-miscible solvent/water composition advantageously removes many undesirable non-protein components (pigments, mycotoxins, carbohydrates (such as sugars), organic acids, lipids, etc.) from the starting destarched corn gluten meal.

In an aspect, the refined, destarched corn gluten meal comprises less than about 1.5 wt % oil. In an aspect, the refined, destarched corn gluten meal comprises less than about 1 wt % oil. In an aspect, the refined, destarched corn gluten meal comprises less than about 0.5 wt % oil. In an aspect, the refined, destarched corn gluten meal comprises less than about 0.1 wt % oil. In an aspect, the refined, destarched corn gluten meal has no detectable presence of oil.

As noted above, the starting destarched corn gluten meal 120 may be yellowish-orange in color because most of the corn pigments (luteins, zeaxanthins, cryptoxanthins, and carotenes) concentrate into the protein stream. Corn pigments are known to be fat soluble and have strong affinity to bind to zein protein. Xanthophylls (luteins, zeaxanthins and cryptoxanthins) make up to 94% of total pigment amounts of the starting corn gluten meal. This color is undesirable for most food-grade applications. Accordingly, the solvent-mediated defatting step described herein eliminates a substantial amount of the color and provides in an aspect a refined, destarched corn gluten meal having an “a*” color value in a range from about −0.05 to 1.5, or about −0.6 to 0.5, or about −0.5 to 0.5, or about −0.4 to 0.5, or about −0.3 to 0.5, or about −0.2 to 0.5, or about −0.1 to 0.5. In aspects, the “a*” color value may be in a range from about −0.6 to 0.3, or about −0.5 to 0.3, or about −0.4 to about 0.3, or about −0.3 to 0.3, or about −0.2 to 0.3, or about −0.1 to 0.3. In aspects, the “a*” value may range from about −0.6 to −0.1, or about −0.6 to −0.2, or about −0.5 to −0.1, or about −0.5 to −0.2. Further, the in an aspect refined, destarched corn gluten meal may have a “b*” color value in a range from about 10 to about 25, or about 10 to about 22, or about 10 to 20. In aspects, the “b*” value may range from about 10 to 16, or about 10 to 15, or about 10 to 14, or about 10 to 13. Further, in an aspect a refined, destarched corn gluten meal may have a “L*” color value ranging from about 88 and 95, or about 89 to 95, or about 90 to 95. In aspects, the “L*” color value may range from about 88 to 92, or about 89 to 92, or about 90 to 92. The color values provided herein correspond to a refined, destarched corn gluten meal that is off-white in appearance.

In an aspect, the refined, destarched corn gluten meal has a total soluble carbohydrate concentration of about 40 gram/kilogram (g/kg) or less. In an aspect, the refined, destarched corn gluten meal has total soluble carbohydrate concentration of about 30 g/kg or less. In an aspect, the refined, destarched corn gluten meal has total soluble carbohydrate concentration of about 25 g/kg or less. In an aspect, the refined, destarched corn gluten meal has total soluble carbohydrate concentration of about 20 g/kg or less. In an aspect, the refined, destarched corn gluten meal has total soluble carbohydrate concentration of or about 10 g/kg or less.

In an aspect, the refined, destarched corn gluten meal comprises at least about 86 wt %; or at least about 87 wt %; or at least about 88 wt %; or at least about 89 wt %; or at least about 90 wt %; or at least about 91 wt %; or at least about 92 wt %, corn protein on a dry basis (db).

In an aspect, the refined, destarched corn gluten meal has an aflatoxin level of less than 2.0 part per billion (ppb), less than about 1.5 ppb, less than about 1 ppb, less than about 0.5 ppb, or no detectable presence of aflatoxin; a zearalenone level of less than about 200 ppb, less than about 150 ppb, less than 100 ppb, less than 50 ppb, less than 10 ppb, less than 5 ppb, less than about 1 ppb, less than about 0.5 ppb, less than 0.1 ppb, or no detectable presence of zearalenone; a deoxynivalenol level of less than 1 part per million (ppm), less than about 0.5 ppm, less than about 0.1 ppm, or no detectable presence of deoxynivalenol; and a fumonisin level of less than about 4 ppm, less than about 3 ppm, less than about 2 ppm, less than about 1 ppm, less than about 0.5 ppm, or no detectable presence of fumonisin; and mixtures thereof.

In an aspect, the refined, destarched corn gluten meal has less than about 1.5% oil on a dry basis; has an L* color value of from about 88 to 95, an “a*” color value ranging from about −0.5 to 1.5, and a “b*” color value ranging from about 10 to 25; has a soluble carbohydrate concentration of 40 g/kg or less; and comprises at least about 85 wt % corn protein on a dry basis.

Suitable general processes for production of refined corn protein products have been previously described that may be adapted to provide the present refined, destarched corn gluten meal. See, for example, pending US Patent Applications 2018/0118780 to Chen et al.; 2019/116851 to Porter et al.; and 2019/297915 to Frank et al.; which are hereby incorporated by reference in their entirety.

In an aspect, the destarched corn gluten meal is defatted with a water-miscible solvent/water defatting composition comprising from about 85 wt % to about 99 wt % solvent. In an aspect, the destarched corn gluten meal is defatted with a water-miscible solvent/water defatting composition comprising from about 90 wt % to about 98 wt % solvent. In an aspect, the destarched corn gluten meal is defatted with a water-miscible solvent/water defatting composition comprising from about 95 wt % to about 97.5 wt % solvent.

In an aspect, a series of solvent-mediated defatting steps may be performed. A benefit to the processes described herein is the reduction in solvent use compared to other purification processes described in the prior art. In the processes described herein, about 3 to 40 liters (L) of solvent are used per kilogram (kg) of destarched corn gluten meal to achieve the desired purity of the refined, destarched corn gluten meal.

In an aspect, the destarched corn gluten meal is defatted at temperatures ranging from about 5 to about 50° C.; and in an aspect in a range of from about 20 to about 30° C. to avoid adverse affects on the protein yield and the functional properties of the protein.

The refined, destarched corn gluten meal 130 as described above is then treated in a solvent-mediated fractionation step 131 to recover a zein-enriched protein composition 140 and a zein-depleted protein composition 150. In an aspect, the solvent-fractionation step 131 comprises treatment of the refined, destarched corn gluten meal 130 with an ethanol/water extraction composition comprising a 55-80 wt % ethanol/water solution. In an aspect, the solvent-fractionation step 131 comprises treatment of the refined, destarched corn gluten meal 130 with an ethanol/water extraction composition comprising a 60-75 wt % ethanol/water solution. In an aspect, the solvent-fractionation step 131 comprises treatment of the refined, destarched corn gluten meal 130 with an ethanol/water extraction composition comprising a 60-70 wt % ethanol/water solution. In an aspect, the solvent-fractionation step 131 comprises treatment of the refined, destarched corn gluten meal 130 with an ethanol/water extraction composition comprising a 65 wt % ethanol/water solution. In this treatment, a zein-enriched protein composition is extracted from the refined, destarched corn gluten meal in the supernatant, leaving behind a zein-depleted protein composition as the extracted solids (residue). The separation of the supernatant from the extracted solids is carried out by one or more solids-liquid separation steps, such as filtration or centrifugation, and homogenization techniques commonly known to one skilled in the art.

In an aspect, ethanol/water extraction compositions having higher ethanol concentrations may increase the amount of protein present in the zein-depleted protein composition. Furthermore, it has been found that using destarched corn gluten meal yields a purer protein in the zein-enriched protein composition than corn gluten meal. In an aspect, the zein-depleted protein composition can have up to 1.75 times higher protein concentration when prepared from a zein-enriched protein composition as described herein as compared to a like composition prepared from corn gluten meal.

In an aspect, the zein-enriched protein composition 140 comprises at least 50% of the protein present in the refined, destarched corn gluten meal 130. In an aspect, the zein-enriched protein composition 140 comprises at least 55% of the protein present in the refined, destarched corn gluten meal 130.

In an aspect, the zein-depleted protein composition 150 comprises at least 30% of the protein present in the refined, destarched corn gluten meal 130. In an aspect, the zein-depleted protein composition 150 comprises at least 40% of the protein present in the refined, destarched corn gluten meal 130.

Specific methods for obtaining a zein-enriched fraction and a zein-depleted fraction, and the corn protein compositions so obtained are described in WO2019060673A1, the disclosure of which is incorporated by reference herein. However, it should be noted that the process as described in WO2019060673A1 is applied to gluten meal (“CGM”) or destarched CGM (i.e. the destarched CGM described in U.S. Pat. No. 9,226,515 and hereinafter referred to as Empyreal®), but the process for obtaining a zein-enriched hydrolysate fraction and a zein-depleted hydrolysate fraction was not previously applied to refined, destarched corn gluten meal as described herein.

In an aspect, the zein-enriched protein composition comprises from 75 wt % to 95 wt % (dry solids) protein. In an aspect, the zein-enriched protein composition comprises from 78 wt % to 83 wt % (dry solids) protein.

In an aspect, the zein-depleted protein composition comprises from 50 wt % to 80 wt % (dry solids) protein. In an aspect, the zein-depleted protein composition comprises from 70 wt % to 80 wt % (dry solids) protein.

The zein-enriched protein composition 140 and the zein-depleted protein composition 150 have different amino acid distributions. Additionally, the zein-enriched protein composition 140 and the zein-depleted protein composition 150 have different fatty acid profiles.

The zein-enriched protein composition 140 and the zein-depleted protein composition 150 are separately hydrolyzed by enzyme hydrolysis 151 under controlled pH and temperature conditions. The hydrolysis of the zein-enriched protein composition 140 is terminated to provide a zein-enriched protein hydrolysate composition 160 having a protein solubility of from about 15% to about 20% at a pH selected from the group consisting of pH 3.4, pH 7.0, and of both pH 3.4 and pH 7.0. The hydrolysis of the zein-depleted protein suspension is terminated to provide a zein-depleted protein hydrolysate composition 170 having a protein solubility of from about 20% to about 35% at a pH selected from the group consisting of pH 3.4, pH 7.0, and of both pH 3.4 and pH 7.0.

This hydrolysis is carried out as follows:

In an aspect, the zein-enriched protein composition 140 and/or separately the zein-depleted protein composition 150 is added to a solvent system comprising water at a temperature and in an amount suitable to provide a zein-enriched protein suspension (or separately a zein-depleted protein suspension). In an aspect, the zein-enriched protein composition (or separately the zein-depleted protein composition) is added to the solvent system at a temperature of from about 40° C. to about 70° C. In an aspect, the zein-enriched protein composition (or separately the zein-depleted protein composition) is added to a solvent system at a temperature of from about 45° C. to about 55° C. In an aspect, the zein-enriched protein composition (or separately the zein-depleted protein composition) is added to the solvent system in an amount to obtain a corn protein suspension having a solids content of from about 1% (w/v) to about 30% (w/v). In an aspect, the zein-enriched protein composition (or separately the zein-depleted protein composition) is added to the solvent system in an amount to obtain a corn protein suspension having a solids content of from about 1% (w/v) to about 25% (w/v). In an aspect, the zein-enriched protein composition (or separately the zein-depleted protein composition) is added to the solvent system in an amount to obtain a corn protein suspension having a solids content of from about 15% (w/v) to about 25% (w/v). In an aspect, the zein-enriched protein composition (or separately the zein-depleted protein composition) is added to the solvent system in an amount to obtain a corn protein suspension having a solids content of from about 20% (w/v) to about 35% (w/v). In an aspect, the zein-enriched protein composition (or separately the zein-depleted protein composition) is added to the solvent system in an amount to obtain from about 1 to about 15% (w/v) corn protein suspension. In an aspect, the zein-enriched protein composition (or separately the zein-depleted protein composition) is added to the solvent system in an amount to obtain from about 3 to about 8% (w/v) corn protein suspension. In an aspect, the zein-enriched protein composition (or separately the zein-depleted protein composition) is added to the solvent system in an amount to obtain about a 5% (w/v) corn protein suspension.

An enzyme is added to the corn protein suspension (either zein-enriched or zein-depleted) containing the zein-enriched protein composition (or separately the zein-depleted protein composition) at a ratio of from about 1:100 to about 1:20 by weight of enzyme to corn protein. In an aspect, the pH of the corn protein suspension is adjusted and/or maintained at a desired level prior to addition of the enzyme. In an aspect, the pH of the corn protein suspension is adjusted and/or maintained at from about 5.0 to about 6.0 prior to addition of the enzyme.

In an aspect, the enzyme can be added at a ratio from about 1:55 to about 1:20 (by weight) of enzyme to corn protein. In an aspect, the enzyme can be added at a ratio from about 1:55 to about 1:45 (by weight) of enzyme to corn protein. In an aspect, the enzyme can be added at a ratio from about 1:30 to about 1:20 (by weight) of enzyme to corn protein. In an aspect, the enzyme can be added at a ratio from about 1:50 to about 1:25 (by weight) of enzyme to corn protein. In an aspect, the ratio of enzyme to corn protein is about 1:50. In an aspect, the ratio of enzyme to corn protein is about 1:37.5. In an aspect, the ratio of enzyme to corn protein is about 1:25. In an aspect, the ratio of enzyme to corn protein is about 1:100.

The term “enzyme” means a composition having an active enzyme product. One skilled in the art will appreciate enzyme activity and inclusion level can be varied within an enzyme product. In an aspect, the enzyme is a protease. In an aspect, the protease enzyme is obtained from a fungus. In an aspect, the protease is obtained from the fungus Aspergillus oryzae. In an example, the fungal enzyme can be Protease M “Amano” SD from Amano Enzyme Inc. While not being bound by theory, it is believed that fungal enzymes in particular when used in the hydrolysis process as described herein targets specific sites on the protein resulting in the release of hydrophilic peptides that are not perceived as bitter, and may when used under the conditions as described herein minimize protein off-flavor.

In an aspect, the pH and temperature of the corn protein suspension (either zein-enriched or zein-depleted) containing the enzyme is controlled for a time sufficient to hydrolyze the corn protein to the desired degree of hydrolysis. In an aspect, the pH of the corn protein suspension during hydrolysis is from about 5.0 to about 6.0. In an aspect, the pH of the corn protein suspension during hydrolysis is about 5.5. In an aspect, the temperature of the corn protein suspension during hydrolysis is from about 40° C. to about 70° C. In an aspect, the temperature of the corn protein suspension during hydrolysis is from about 45° C. to about 55° C.; or from about 50° C. to about 60° C. In an aspect, the temperature of the corn protein suspension during hydrolysis is about 50° C. In an aspect, the hydrolysis of the corn protein suspension is carried out for a time of from about 15 minutes to about 180 minutes. In an aspect, the hydrolysis of the corn protein suspension is carried out for a time of from about 30 minutes to about 120 minutes. In an aspect, the hydrolysis of the corn protein suspension is carried out for a time of from about 45 minutes to about 90 minutes.

In an aspect, the hydrolysis of the zein-enriched protein suspension is terminated when the corn protein hydrolysate composition has a degree of hydrolysis of from about 5.5% to about 8%. In an aspect, the hydrolysis of the corn protein suspension is carried out with monitoring of the degree of hydrolysis to determine the point at which the hydrolysis is to be suspended. In an aspect, the hydrolysis of the corn protein suspension is carried out for a time determined to be predictive of achieving the desired degree of hydrolysis of the composition.

In an aspect, the pH and temperature of the zein-enriched protein suspension containing the enzyme is controlled for a time sufficient to hydrolyze the corn protein so that the zein-enriched protein hydrolysate composition has a solubility of from about 15% to about 20% at a pH selected from the group consisting of pH 3.4, pH 7.0, and of both pH 3.4 and pH 7.0. In an aspect, the pH and temperature of the zein-enriched protein suspension containing the enzyme is controlled for a time sufficient to hydrolyze the corn protein so that the refined zein-enriched protein hydrolysate composition has a solubility of from about 16% to about 20% at a pH selected from the group consisting of pH 3.4, pH 7.0, and of both pH 3.4 and pH 7.0. In an aspect, the hydrolysis of the corn protein suspension is carried out with monitoring of the solubility of the composition to determine the point at which the hydrolysis is to be suspended. In an aspect, the hydrolysis of the corn protein suspension is carried out for a time determined to be predictive of achieving the desired solubility of the composition.

In an aspect, the pH and temperature of the zein-depleted protein suspension containing the enzyme is controlled for a time sufficient to hydrolyze the corn protein so that the zein-depleted protein hydrolysate composition has a solubility of from about 20% to about 35% at a pH selected from the group consisting of pH 3.4, pH 7.0, and of both pH 3.4 and pH 7.0. In an aspect, the pH and temperature of the zein-depleted protein suspension containing the enzyme is controlled for a time sufficient to hydrolyze the corn protein so that the refined zein-depleted protein hydrolysate composition has a solubility of from about 22% to about 32% at a pH selected from the group consisting of pH 3.4, pH 7.0, and of both pH 3.4 and pH 7.0.

In an aspect, the hydrolysis of the zein-enriched protein suspension is terminated when the refined zein-enriched protein hydrolysate composition has a degree of hydrolysis of from about 3% to about 5%.

In an aspect, the hydrolysis of the zein-depleted protein suspension is terminated when the refined zein-depleted protein hydrolysate composition has a degree of hydrolysis of from about 5.5% to about 9%. In an aspect, the hydrolysis of the zein-depleted protein suspension is terminated when the refined zein-depleted protein hydrolysate composition has a degree of hydrolysis of from about 5.5% to about 7%.

In an aspect, the hydrolysis of the zein-enriched protein suspension and/or the zein-depleted protein suspension is terminated by adjusting the pH to 7 and then heating at 75° C. for 5 min. The hydrolysates are dried afterwards, for example, by freeze-drying, spray drying or other common drying methods. The dried powder is then subjected to solubility tests and degree of hydrolysis.

Specific methods for enzyme treatment of refined, destarched corn gluten meal are described in PCT/US2019/059482 filed on Nov. 1, 2019, the disclosure of which is incorporated by reference herein. However, it should be noted that the process as described in PCT/US2019/059482 is applied to refined, destarched corn gluten meal, but not to zein-enriched and/or a zein-depleted fractions as described herein.

In an aspect, the conditions of hydrolysis of the corn protein suspension (either zein-enriched or zein-depleted) are controlled to select a desired degree of hydrolysis and additionally to provide a high level of solubility of the resulting hydrolyzed corn protein suspension for that given degree of hydrolysis. It has been found that selection of the degree of hydrolysis in combination with choosing hydrolysis conditions that provide a high level of solubility of the resulting hydrolyzed corn protein suspension provides unique zein-enriched protein hydrolysate compositions or zein-depleted protein hydrolysate compositions having particular component profiles that afford distinctive nutritional and/or performance properties. For purposes of the present discussion, the distinctiveness of any given properties of the zein-enriched protein hydrolysate compositions or zein-depleted protein hydrolysate compositions (or fractions thereof) is determined by comparison with like compositions prepared using a different selection of one or both of the degree of hydrolysis and solubility property parameters of the resulting hydrolyzed corn protein suspension.

In an aspect, the zein-enriched protein hydrolysate compositions or zein-depleted protein hydrolysate compositions as described herein exhibit distinctive unique flavor profiles as a result of the particular component profiles obtained by the select a desired degree of hydrolysis and solubility range of the resulting hydrolyzed corn protein suspension.

In an aspect, the zein-enriched protein hydrolysate compositions or zein-depleted protein hydrolysate compositions as described herein exhibit distinctive nutrition profiles as a result of the particular component profiles obtained by the selection of a desired degree of hydrolysis and solubility range of the resulting hydrolyzed corn protein suspension.

In an aspect, the zein-enriched protein hydrolysate compositions or zein-depleted protein hydrolysate compositions as described herein exhibit distinctive characteristics in handling properties and/or mixing with other materials, such as food or beverage components, as a result of the particular component profiles obtained by the selection of a desired degree of hydrolysis and solubility range of the resulting hydrolyzed corn protein suspension. In an aspect, the zein-enriched protein hydrolysate compositions or zein-depleted protein hydrolysate compositions exhibit distinctive characteristics in ability to form a foam, or in ability to form a gel or emulsification properties.

In an aspect, the conditions of hydrolysis of the zein-enriched corn protein suspension are controlled to select a degree of hydrolysis of from about 10% to about 30% and additionally to select a solubility range of the resulting hydrolyzed zein-enriched corn protein suspension of from about 25% to about 40% at pH 7. In an aspect, the conditions of hydrolysis of the zein-enriched corn protein suspension are controlled to select a degree of hydrolysis of from about 12% to about 25% and additionally to select a solubility range of the resulting hydrolyzed zein-enriched corn protein suspension of from about 25% to about 38% at pH 7. In an aspect, the conditions of hydrolysis of the zein-enriched corn protein suspension are controlled to select a degree of hydrolysis of from about 15% to about 20% and additionally to select a solubility range of the resulting hydrolyzed zein-enriched corn protein suspension of from about 28% to about 35% at pH 7.

In an aspect, the conditions of hydrolysis of the zein-enriched corn protein suspension are controlled to select a degree of hydrolysis of from about 2% to about 12% and additionally to select a solubility range of the resulting hydrolyzed zein-enriched corn protein suspension of from about 10% to about 28% at pH 7. In an aspect, the conditions of hydrolysis of the zein-enriched corn protein suspension are controlled to select a degree of hydrolysis of from about 3% to about 10% and additionally to select a solubility range of the resulting hydrolyzed zein-enriched corn protein suspension of from about 12% to about 25% at pH 7. In an aspect, the conditions of hydrolysis of the zein-enriched corn protein suspension are controlled to select a degree of hydrolysis of from about 4% to about 8% and additionally to select a solubility range of the resulting hydrolyzed zein-enriched corn protein suspension of from about 15% to about 28% at pH 7.

In an aspect, the conditions of hydrolysis of the zein-depleted corn protein suspension are controlled to select a degree of hydrolysis of from about 12% to about 25% and additionally to select a solubility range of the resulting hydrolyzed zein-depleted corn protein suspension of from about 40% to about 68% at pH 7. In an aspect, the conditions of hydrolysis of the zein-depleted corn protein suspension are controlled to select a degree of hydrolysis of from about 14% to about 22% and additionally to select a solubility range of the resulting hydrolyzed zein-depleted corn protein suspension of from about 42% to about 65% at pH 7. In an aspect, the conditions of hydrolysis of the zein-depleted corn protein suspension are controlled to select a degree of hydrolysis of from about 16% to about 20% and additionally to select a solubility range of the resulting hydrolyzed zein-depleted corn protein suspension of from about 45% to about 55% at pH 7.

In an aspect, the conditions of hydrolysis of the zein-depleted corn protein suspension are controlled to select a degree of hydrolysis of from about 3% to about 15% and additionally to select a solubility range of the resulting hydrolyzed zein-depleted corn protein suspension of from about 10% to about 40% at pH 7. In an aspect, the conditions of hydrolysis of the zein-depleted corn protein suspension are controlled to select a degree of hydrolysis of from about 3% to about 12% and additionally to select a solubility range of the resulting hydrolyzed zein-depleted corn protein suspension of from about 15% to about 35% at pH 7. In an aspect, the conditions of hydrolysis of the zein-depleted corn protein suspension are controlled to select a degree of hydrolysis of from about 5% to about 10% and additionally to select a solubility range of the resulting hydrolyzed zein-depleted corn protein suspension of from about 20% to about 30% at pH 7.

In an aspect, the conditions of hydrolysis of the corn protein suspension (either zein-enriched or zein-depleted) are controlled by selection of the pH of the corn protein suspension during hydrolysis, the temperature of the corn protein suspension during hydrolysis, the time of the hydrolysis reaction, the selection of the enzyme used in the hydrolysis reaction, and the ratio of enzyme to substrate used in the hydrolysis reaction.

In an aspect, a zein-enriched corn protein suspension having a selected high degree of hydrolysis is prepared by carrying out an enzymatic hydrolysis at a pH of from about 5 to 5.5, at a temperature of from about 48 to 55° C., for a time of from about 100 to 140 minutes, and at an enzyme to substrate ratio of from about 1:20 to about 1:30. In an aspect, this hydrolysis provides a zein-enriched corn protein suspension having a selected high degree of hydrolysis that is from about 10% to about 30%. In an aspect, this hydrolysis provides a zein-enriched corn protein suspension having a selected high degree of hydrolysis that is from about 12% to about 25%

In an aspect, this hydrolysis provides a zein-enriched corn protein suspension having a selected high degree of hydrolysis that is from about 15% to about 20%.

In an aspect, a zein-enriched corn protein suspension having a selected low degree of hydrolysis is prepared by carrying out an enzymatic hydrolysis at a pH of from about 5 to 5.5, at a temperature of from about 53 to 60° C., for a time of from about 50 to 90 minutes, and at an enzyme to substrate ratio of from about 1:40 to about 1:70. In an aspect, this hydrolysis provides a zein-enriched corn protein suspension having a selected low degree of hydrolysis that is from about 2% to about 10%. In an aspect, this hydrolysis provides a zein-enriched corn protein suspension having a selected low degree of hydrolysis that is from about 4% to about 8%. In an aspect, this hydrolysis provides a zein-enriched corn protein suspension having a selected low degree of hydrolysis that is from about 5% to about 8%.

In an aspect, a zein-depleted corn protein suspension having a selected high degree of hydrolysis is prepared by carrying out an enzymatic hydrolysis at a pH of from about 5 to 5.5, at a temperature of from about 48 to 53° C., for a time of from about 100 to 140 minutes, and at an enzyme to substrate ratio of from about 1:20 to about 1:30. In an aspect, this hydrolysis provides a zein-depleted corn protein suspension having a selected high degree of hydrolysis that is from about 10% to about 30%. In an aspect, this hydrolysis provides a zein-depleted corn protein suspension having a selected high degree of hydrolysis that is from about 12% to about 25%

In an aspect, this hydrolysis provides a zein-depleted corn protein suspension having a selected high degree of hydrolysis that is from about 15% to about 20%.

In an aspect, a zein-depleted corn protein suspension having a selected low degree of hydrolysis is prepared by carrying out an enzymatic hydrolysis at a pH of from about 5.5 to 6.2, at a temperature of from about 45 to 55° C., for a time of from about 45 to 75 minutes, and at an enzyme to substrate ratio of from about 1:25 to about 1:55. In an aspect, this hydrolysis provides a zein-depleted corn protein suspension having a selected low degree of hydrolysis that is from about 2% to about 10%. In an aspect, this hydrolysis provides a zein-depleted corn protein suspension having a selected low degree of hydrolysis that is from about 4% to about 8%. In an aspect, this hydrolysis provides a zein-depleted corn protein suspension having a selected low degree of hydrolysis that is from about 5% to about 8%.

Further separation of the water-soluble proteins from the water-insoluble proteins in the refined zein-enriched protein hydrolysate composition provides unique water-soluble refined zein-enriched protein hydrolysate compositions and unique water-insoluble refined zein-enriched protein hydrolysate compositions, each of which have particular component profiles that afford distinctive nutritional and/or performance properties as discussed above.

Likewise, further separation of the water-soluble proteins from the water-insoluble proteins in the refined zein-depleted protein hydrolysate composition provides unique water-soluble refined zein-depleted protein hydrolysate compositions and unique water-insoluble refined zein-depleted protein hydrolysate compositions, each of which have particular component profiles that afford distinctive nutritional and/or performance properties as discussed above.

The particular component profiles of these four unique separated protein hydrolysate compositions are advantageously even more strongly differentiated by controlling the conditions of hydrolysis of the corn protein suspension (either zein-enriched or zein-depleted) to select a desired degree of hydrolysis and additionally to provide a high level of solubility of the resulting hydrolyzed corn protein suspension for that given degree of hydrolysis as described herein. The resulting compositions may be selected for specific uses with particular advantage due to the unique component profiles of each of the compositions.

In an aspect, the refined zein-enriched protein hydrolysate composition 160 is then separated into a water-soluble refined zein-enriched protein hydrolysate composition 180 and a water-insoluble refined zein-enriched protein hydrolysate composition 185 by a water-mediated fractionation step 171. In an aspect, the water-mediated fractionation step 171 comprises treatment of the refined zein-enriched protein hydrolysate composition 160 with a water extraction composition. In this treatment, a water-soluble refined zein-enriched protein hydrolysate composition 180 is extracted from the refined zein-enriched protein hydrolysate composition 160 in the supernatant, leaving behind a water-insoluble refined zein-enriched protein hydrolysate composition 185 as the extracted solids (residue). The separation of the supernatant from the extracted solids is carried out by one or more solids-liquid separation steps, such as filtration or centrifugation techniques, commonly known to one skilled in the art.

In an aspect, the refined zein-depleted protein hydrolysate composition 170 is then separated into a first zein-depleted composition comprising water-soluble refined zein-depleted protein hydrolysate composition 190 and a water-insoluble refined zein-depleted protein hydrolysate composition 195 by a water-mediated fractionation step 171. In an aspect, the water-mediated fractionation step 171 comprises treatment of the refined zein-depleted protein hydrolysate composition 170 with a water extraction composition. In this treatment, a water-soluble refined zein-depleted protein hydrolysate composition 190 is extracted from the refined zein-depleted protein hydrolysate composition 170 in the supernatant, leaving behind a water-insoluble refined zein-depleted protein hydrolysate composition 195 as the extracted solids (residue). The separation of the supernatant from the extracted solids is carried out by one or more solids-liquid separation steps, such as filtration or centrifugation, and homogenization techniques commonly known to one skilled in the art.

In an aspect of the present method, the refined zein-enriched protein hydrolysate composition 160; and/or the refined zein-depleted protein hydrolysate composition 170; and/or the water-soluble refined zein-enriched protein hydrolysate composition 180; and/or the water-insoluble refined zein-enriched protein hydrolysate composition 185; and/or the water-soluble refined zein-depleted protein hydrolysate composition 190; and/or a water-insoluble refined zein-depleted protein hydrolysate composition 195 are dried. In an aspect, the hydrolysate compositions noted above are dried by freeze-drying or spray-drying. One skilled in the art will recognize that other drying methods or equipment may be suitable for drying these products. In an aspect, the hydrolysate compositions are in the form of a powder. In an aspect, the hydrolysate compositions are in the form of dried particulates that are ground and sieved to a size of less than 300 μm. In an aspect, the hydrolysate compositions are in the form of dried particulates that are ground and sieved to a size of less than 100 μm. In an aspect, the hydrolysate compositions are in the form of dried particulates that are ground and sieved to a size of less than 50 μm. In an aspect, the hydrolysate compositions have a moisture content of less than 10%. In an aspect, the hydrolysate compositions contain at least about 75 wt % corn protein. In an aspect, the hydrolysate compositions contain at least about 79 wt % corn protein. In an aspect, the hydrolysate compositions contain at least about 80 wt % corn protein. In an aspect, the hydrolysate compositions contain at least about 85 wt % corn protein. In an aspect, the hydrolysate compositions contain at least about 90 wt % corn protein.

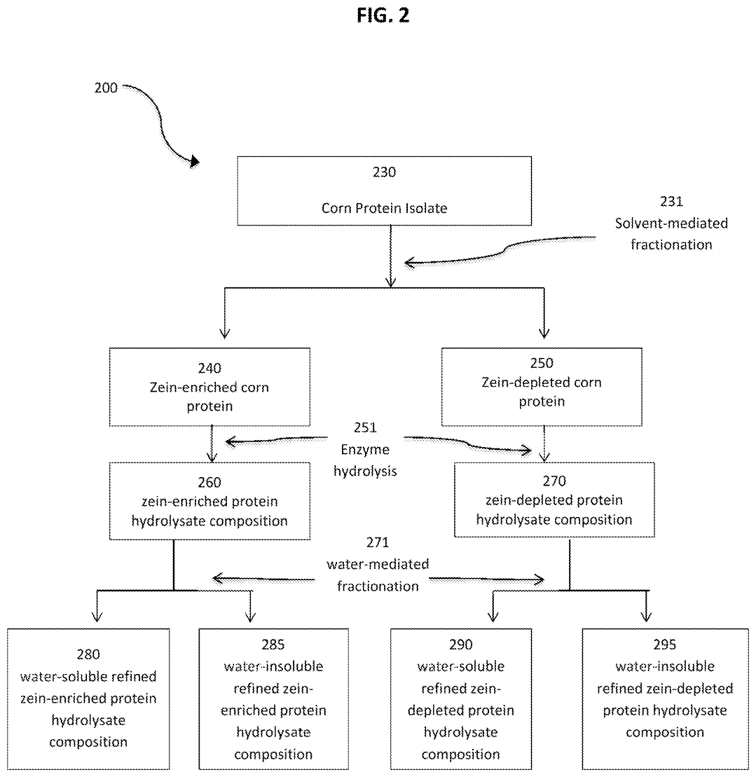

FIG. 2 shows an exemplary process 200 for preparing a refined zein-enriched protein hydrolysate composition and/or a refined zein-depleted protein hydrolysate composition, and optionally further preparing water-soluble and/or water-insoluble compositions from the refined zein-enriched protein hydrolysate composition and/or the refined zein-depleted protein hydrolysate compositions, wherein the starting material is instead described as a corn Protein Isolate Composition 230. In this process, the Protein Isolate Composition 230 is a corn protein composition that has a corn protein concentration of at least 85% (ds), wherein from about 40% to about 70% of the corn protein is soluble in a 65% ethanol/water solution at a temperature of 60° C.

In an aspect, the Protein Isolate Composition 230 is a corn protein composition that

i) comprises less than about 2 wt % oil on a dry basis;

ii) has an L* color value of from about 88 to 95, an “a*” color value ranging from about −0.5 to 1.5, and a “b*” color value ranging from about 10 to 25;

iii) has a soluble carbohydrate concentration of 40 g/kg or less;

iv) comprises at least about 85 wt % protein on a dry basis; and

v) comprises from about 40 to about 70% of a protein that is soluble in a 65% ethanol/water solution at a temperature of 60° C.

In an aspect, the Protein Isolate Composition 230 comprises from about 45 to about 65% of a protein that is soluble in a 65 wt % ethanol/water solution at a temperature of 60° C. In an aspect, the Protein Isolate Composition 230 comprises from about 50 to about 60% of a protein that is soluble in a 65 wt % ethanol/water solution at a temperature of 60° C.

The Protein Isolate Composition 230 as described above is then treated in a solvent-mediated fractionation step 231 to recover a zein-enriched protein composition 240 and a zein-depleted protein composition 250. The solvent-mediated fractionation step 231 is carried out in the same manner to obtain generally the same products as described above in the solvent-fractionation step 131. Likewise, the enzyme hydrolysis step 251 and the water-mediated fractionation step 271 are carried out in the same manner to obtain generally the same products as described above in the enzyme hydrolysis step 151 and the water-mediated fractionation step 171.

In general, hydrolyzed proteins often have a bitter or astringent taste. In an aspect, the refined zein-enriched protein hydrolysate compositions and/or the refined zein-depleted protein hydrolysate compositions (either before or after separation of into water-soluble and water-insoluble compositions) of the present disclosure may be formulated to not have a bitter flavor.

In an aspect, the refined zein-enriched protein hydrolysate compositions and/or the refined zein-depleted protein hydrolysate compositions (either before or after separation of into water-soluble and water-insoluble compositions) can be provided in the form of a solution or slurry. In an aspect the protein hydrolysate compositions described above can be provided in the form of a concentrated solution, paste, or slurry, e.g., having a solids content of from about 40 wt % to about 80 wt % solids, or having a solids content of from about 40 wt % to about 60 wt % solids. Providing the protein hydrolysate compositions in liquid form provides handling advantages, such as ease in addition and mixing of the hydrolysate into a liquid, and avoidance of challenges of handling powders. In an aspect the protein hydrolysate compositions are provided in the form of a solution, paste, or slurry in aseptic packaging. In an aspect, the protein hydrolysate compositions can be available in a powder form. The powder composition of the protein hydrolysate compositions can contain less than 100% corn protein.

In an aspect, the protein hydrolysate compositions can contain at least about 70 wt % corn protein (ds). In an aspect, the protein hydrolysate compositions can contain at least about 75 wt % corn protein. In an aspect, the protein hydrolysate compositions can contain at least about 80 wt % corn protein. In an aspect, the protein hydrolysate compositions can contain about 79 wt % corn protein. In an aspect, the protein hydrolysate compositions can contain about 80 wt % corn protein. In an aspect, the protein hydrolysate compositions have a moisture content of less than 10%.

In an aspect, a refined zein-enriched protein hydrolysate composition is provided, wherein the composition:

i) has a protein solubility of from about 15% to about 20% at a pH selected from the group consisting of pH 3.4, pH 7.0, and of both pH 3.4 and pH 7.0, and

ii) has a protein concentration of from about 75% to about 95%.

In an aspect, a refined zein-depleted protein hydrolysate composition is provided, wherein the composition:

i) has a protein solubility of from about 20% to about 35% at a pH selected from the group consisting of pH 3.4, pH 7.0, and of both pH 3.4 and pH 7.0, and

ii) has a protein concentration of from about 75% to about 95%.

In an aspect, a water-soluble refined zein-enriched protein hydrolysate composition is provided, wherein the composition:

i) has a protein solubility of from about 95% to 100% at a pH selected from the group consisting of pH 3.4, pH 7.0, and of both pH 3.4 and pH 7.0,

ii) has a protein concentration of from about 50% to about 75%, and

iii) is water-soluble.

In an aspect, a water-insoluble refined zein-enriched protein hydrolysate composition is provided, wherein the composition:

i) has a protein solubility of from 0% to about 5% at a pH selected from the group consisting of pH 3.4, pH 7.0, and of both pH 3.4 and pH 7.0,

ii) has a protein concentration of from about 80% to 100%, and

iii) is water-insoluble.

In an aspect, a water-soluble refined zein-depleted protein hydrolysate composition is provided, wherein the composition:

i) has a protein solubility of from about 95% to 100% at a pH selected from the group consisting of pH 3.4, pH 7.0, and of both pH 3.4 and pH 7.0,

ii) has a protein concentration of from about 75% to about 95%, and

iii) is water-soluble.

In an aspect, a water-insoluble refined zein-depleted protein hydrolysate composition is provided, wherein the composition:

i) has a protein solubility of from 0% to about 5% at a pH selected from the group consisting of pH 3.4, pH 7.0, and of both pH 3.4 and pH 7.0,

ii) has a protein concentration of from about 75% to about 95%, or has a protein concentration of from about 65% to about 85%, and

iii) is water-insoluble.

In an aspect, a water-insoluble refined zein-depleted protein hydrolysate composition is provided, wherein the composition:

i) has a protein solubility of from 0% to about 5% at a pH selected from the group consisting of pH 3.4, pH 7.0, and of both pH 3.4 and pH 7.0,

ii) has a protein concentration of from about 65% to about 85%, and

iii) is water-insoluble.

In an aspect, a refined zein-enriched protein hydrolysate composition is provided, wherein the composition

i) has a protein degree of hydrolysis of from about 2% to about 12%;

ii) has a protein solubility of from about 10% to about 28% at pH 7, and

iii) has a protein concentration of from about 75% to about 95%.

In an aspect, a refined zein-enriched protein hydrolysate composition is provided, wherein the composition

i) has a protein degree of hydrolysis of from about 10% to about 30%;

ii) has a protein solubility of from about 25% to about 40% at pH 7, and

iii) has a protein concentration of from about 75% to about 95%.

In an aspect, a refined zein-depleted protein hydrolysate composition is provided, wherein the composition

i) has a protein degree of hydrolysis of from about 3% to about 15%;

ii) has a protein solubility of from about 10% to about 40% at pH 7, and

iii) has a protein concentration of from about 75% to about 95%, or has a protein concentration of from about 65% to about 85%.

In an aspect, a refined zein-depleted protein hydrolysate composition is provided, wherein the composition

i) has a protein degree of hydrolysis of from about 12% to about 25%;

ii) has a protein solubility of from about 40% to about 68% at pH 7, and

iii) has a protein concentration of from about 75% to about 95%, or has a protein concentration of from about 65% to about 85%.

In an aspect, water soluble fractions or water insoluble fractions of the above compositions may be provided.

In an aspect, the present process provides a choice of two different corn protein compositions being soluble in water for use in various food and beverage applications and in nutritional supplement applications. Because one of these soluble corn protein compositions is obtained from a zein-enriched protein composition and the other is obtained from a zein-depleted protein composition, the respective soluble corn protein compositions will exhibit different physical properties and will have different amino acid profiles. The soluble corn protein compositions obtained from a zein-enriched protein composition and from a zein-depleted protein composition will therefore provide nutritional benefits that are different from each other, and additionally will have different optimal product uses.

In an aspect, the present process provides a choice of two different corn protein compositions having low solubility in water for use in various non-liquid foods, such as extrusion products (for example, extruded snack products), meat replacement products and nutritional supplement applications. Because one of these low solubility corn protein compositions is obtained from a zein-enriched protein composition and the other is obtained from a zein-depleted protein composition, the respective low solubility corn protein compositions will exhibit different physical properties and will have different amino acid profiles. The low solubility corn protein compositions obtained from a zein-enriched protein composition and from a zein-depleted protein composition will therefore provide nutritional benefits that are different from each other, and additionally will have different optimal product uses.

In an aspect, the present the protein hydrolysate products may be used as a plant protein component for hybrid meat products, and meat replacement products, such as for ground meat products, sausages, and the like.

In an aspect, the corn protein hydrolysate is provided as an ingredient in a food product, such as a beverage or a non-liquid food. In an aspect, the corn protein hydrolysate is present as from about 1 wt % to about 10 wt % of a beverage. In an aspect, the corn protein hydrolysate is present as from about 2 wt % to about 10 wt % of the beverage. In an aspect, the corn protein hydrolysate is present as from about 3 wt % to about 10 wt % of the beverage. In an aspect, the corn protein hydrolysate is present as from about 2 wt % to about 8 wt % of the beverage. In an aspect, the corn protein hydrolysate is present as from about 2 wt % to about 6 wt % of the beverage. In an aspect, the corn protein hydrolysate is present as from about 2 wt % to about 5 wt % of a beverage.

### EXAMPLES

The present patent application will be further described in the following examples, which do not limit the scope of the invention in the claims.

### Example 1: Preparation of Refined Zein-Enriched (ZE) Protein Composition and Refined Zein-Depleted (ZD) Protein Composition

### A. Starting Materials

The protein, fat, organic acids, carbohydrate and ethanol contents of a Corn Protein Isolate, or “CPI” sample is given in Table 1.

TABLE 1
Chemical composition of CPI sample

| Protein | Loss on | Fat | EtOH |
|---|---|---|---|
| (%) as is | Drying | (%) | (<60) |
| 82.0 | 4.66 | 0.25 | 29.1 |

Protein content was determined following the AACCI 46-30.01 Dumas nitrogen combustion method using a nitrogen analyzer (LECO TruSpecN™, St. Joseph, Mich., USA) and a conversion factor of 6.25.

### B. Fractionation of Refined, Destarched Corn Gluten Meal (Corn Protein Isolate “CPI”)

CPI was mixed in 65 wt % ethanol at 15% solids in mass basis at 60 C for 30 min with overhead mixing. The suspension was centrifuged at 5000 rpm for 10 min. The supernatant was poured off and the remaining solids (pellet) were resuspended at the above conditions followed by centrifugation. This step was carried out 3 times in total and all supernatants were combined. The supernatants are rich in zein proteins, and therefor provide the refined zein-enriched (ZE) protein composition. The ethanol was gently removed using rotary evaporation followed by air drying. Similarly, the remaining solids (pellet) are the refined zein-depleted (ZD) protein composition, which comprises mainly albumin, globulin and glutelin proteins. These solids were air-dried. Upon drying, ZE and ZD were ground and sieved (#50 sieve) to less than 300 μm. The protein content of each of the ZE and ZD compositions were determined as mentioned above.

40 g of CPI with 82% protein content (Table 1) was used for fractionation. Upon drying of individual corn protein compositions dry weights were 23.2 g and 17 g for ZE and ZD respectively. The protein contents were 84% for ZE and 78% for ZD. ZE is about 58% of CPI.

### C. Enzymatic Hydrolysis of the Refined Zein-Enriched (ZE) Protein Composition and the Refined Zein-Depleted (ZD) Protein Composition.

1. The refined zein-enriched (ZE) protein composition was mixed for 1 hour in water at 5% protein solution at 50° C. at pH 5.5 (volume of suspension was 100 ml). After pre-hydration, the fungal enzyme, Protease M (Amano Enzyme Inc.) was added at a ratio of enzyme to protein (E:P) of about 1:50 and the pH was adjusted every 10 minutes to maintain the pH. The hydrolysis was then terminated by neutralizing with 1M NaOH and inactivating the enzyme by heating to 75° C. for 5 min. After hydrolysis, the refined zein-enriched protein hydrolysate composition sample was freeze dried.

2. The refined zein-depleted (ZD) protein composition was mixed for 1 hour in water at 5% protein solution at 50° C. at pH 5.5 (volume of suspension was 100 ml). After pre-hydration, the fungal enzyme, Protease M (Amano Enzyme Inc.) was added at a ratio of enzyme to protein (E:P) of about 1:50 and the pH was adjusted every 10 minutes to maintain the pH. The hydrolysis was then terminated by neutralizing with 1M NaOH and inactivating the enzyme by heating to 75° C. for 5 min. After hydrolysis, the refined zein-depleted protein hydrolysate composition sample was freeze dried.

### D. Solubility Analysis

Solubility of the refined zein-enriched protein hydrolysate composition and the refined zein-depleted protein hydrolysate composition was determined at pH 3.4 and 7 with and without thermal treatment (85° C. for 30 minutes). Protein solutions (10 mL at 5 wt % protein) were prepared based on the protein content of the powder (determined by the Dumas method) at pH 3.4 and 7 with continuous stirring for 1 hour. Protein content of 200 μL aliquot was determined by the Dumas method. To evaluate the thermal stability, the samples were heated for 30 min at 85° C. The samples (with or without heat treatment) were centrifuged for 10 minutes at 13,000 rpm and 200 μL of supernatant was analyzed for protein content.

The percent solubility of the protein was calculated based on following equation: (Protein content in the supernatant/protein content before centrifugation)×100=% protein solubility.

The DH of the soluble portion of the protein was determined using OPA method based on (Nielsen, Petersen, & Dambmann, 2001) with minor modifications added for corn. Corn protein (0.01 g) was weighed and mixed with 1 ml of 1% sodium dodecyl sulfate (SDS) and was left overnight at room temperature, while stirring. Later, the sample was centrifuged for 13000 rpm for 10 minutes, and 100 μL of supernatant was diluted 10 times with double distilled water. OPA reagent, serine standard and testing the samples were carried out as described in (Nielsen, Petersen, & Dambmann, 2001).

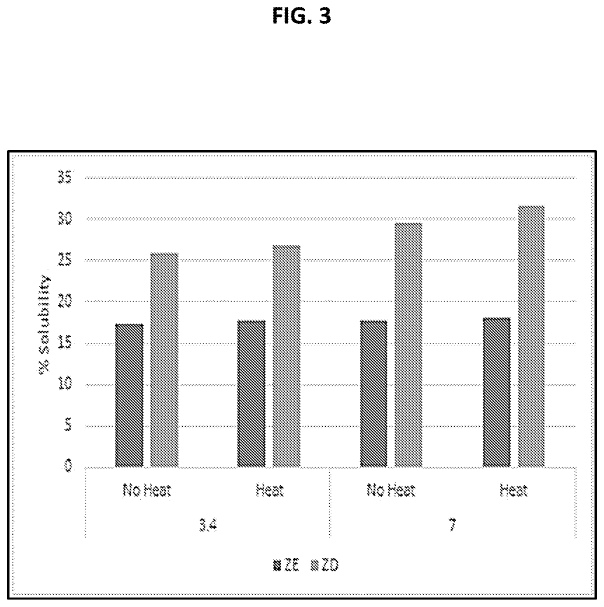

Solubilities of the refined zein-enriched protein hydrolysate compositions and the refined zein-depleted protein hydrolysate compositions with and without heat treatment are shown in FIG. 3. Upon hydrolysis ZE solubility increased up to 17% regardless of the pH tested (3.4 or 7). Also, thermal treatment did not cause any aggregation or reduced solubility. Moreover, ZD solubility was increased up to about 25% when tested at pH 3.4 and about 30% at pH 7. Additionally, thermal treatment seems to further enhance the solubility slightly. Higher solubility seen for ZD could be attributed to higher DH (e.g. 7.8) than that of ZE (e.g. 4.16) giving more hydrophilic (lower net hydrophobicity) peptides.

### Example 2: Amino Acid Composition Analysis of Refined Zein-Enriched and Zein-Depleted Protein Hydrolysate Compositions

The refined zein-enriched protein hydrolysate composition and the refined zein-depleted protein hydrolysate composition prepared in Example 1 were evaluated to determine the respective amino acids content.

A comparison total amino acid content of zein-enriched (ZE) and zein-depleted (ZD) compositions expressed on a g/100 g protein basis is presented in Table 2, below. The ratio of enriched-to-depleted concentration is computed and ratios that differ by more than 20% are highlighted, along with an indicator of the deviation.

TABLE 2

| | ZE-T | ZD-T | |
|---|---|---|---|
| | g/100 g | g/100 g | |
| Amino acid | protein | protein | Enriched/Depleted |
| Aspartic Acid | 5.41 | 5.95 | 0.909 |
| Threonine | 2.84 | 3.62 | 0.784↓ |
| Serine | 4.52 | 3.84 | 1.177 |
| Glutamic Acid | 23.20 | 18.23 | 1.273↑ |
| Proline | 9.10 | 8.46 | 1.075 |
| Glycine | 1.56 | 4.09 | 0.380↓ |
| Alanine | 9.07 | 7.22 | 1.257↑ |
| Cysteine | 1.14 | 2.80 | 0.408↓ |
| Valine | 3.92 | 5.32 | 0.737↓ |
| Methionine | 1.81 | 3.13 | 0.580↓ |
| Isoleucine | 4.14 | 3.86 | 1.073 |
| Leucine | 18.17 | 12.07 | 1.505↑ |
| Tyrosine | 4.70 | 5.09 | 0.924 |
| Phenylalanine | 6.46 | 5.24 | 1.231↑ |
| Lysine | 0.42 | 2.89 | 0.147↓ |
| Histidine | 1.47 | 2.74 | 0.538↓ |
| Arginine | 1.77 | 4.47 | 0.396↓ |
| Tryptophan | 0.31 | 0.97 | 0.315↓ |

Table 2 shows that the initial fractionation into zein-enriched and zein-depleted fractions resulted in compositions having significantly differing amino acid content.

### Example 3—Separation of Refined Zein-Enriched and Zein-Depleted Protein Hydrolysate Compositions into Water-Soluble and Water-Insoluble Compositions

The refined zein-enriched protein hydrolysate composition as prepared in Example 1 was separated into a water-soluble fraction and a water-insoluble fraction by treatment with water. Specifically, the water-soluble refined zein-enriched protein hydrolysate composition is extracted from the refined zein-enriched protein hydrolysate composition in the supernatant, leaving behind a water-insoluble refined zein-enriched protein hydrolysate composition as the extracted solids (residue). 5% (on protein basis) of zein-enriched protein hydrolysate was suspended in water, followed by pH adjustment to 7 and mixing for 1 hour at room temperature. The separation of the supernatant from the extracted solids is carried out by centrifugation. Both the water-soluble fraction and the water-insoluble fraction are dried by freeze-drying for subsequent analysis.

Likewise, the refined zein-depleted protein hydrolysate composition as prepared in Example 1 was separated into a water-soluble fraction and a water-insoluble fraction by treatment with water. Specifically, the water-soluble refined zein-depleted protein hydrolysate composition is extracted from the refined zein-depleted protein hydrolysate composition in the supernatant, leaving behind a water-insoluble refined zein-depleted protein hydrolysate composition as the extracted solids (residue). 5% (on protein basis) of zein-depleted protein hydrolysate was suspended in water, followed by pH adjustment to 7 and mixing for 1 hour at room temperature. The separation of the supernatant from the extracted solids is carried out by centrifugation. Both the water-soluble fraction and the water-insoluble fraction are dried by freeze-drying for subsequent analysis.

### Example 4—Amino Acid Composition Analysis of Water-Soluble Refined Zein-Enriched and Zein-Depleted Protein Hydrolysate Compositions

The water-soluble fractions of the refined zein-enriched protein hydrolysate composition and the refined zein-depleted protein hydrolysate composition prepared in Example 3 were evaluated to determine the respective amino acids content.

A comparison of the total amino acid content of water-soluble zein-enriched (ZE-S) and zein-depleted (ZD-S) compositions expressed on a g/100 g protein basis is presented in Table 3, below. The ratio of enriched-to-depleted concentration is computed and ratios that differ by more than 20% are highlighted, along with an indicator of the deviation.

TABLE 3

| | ZE-S | ZD-S | |
|---|---|---|---|
| | g/100 g | g/100 g | |
| Amino acid | protein | protein | Enriched/Depleted |
| Aspartic Acid | 6.17 | 6.69 | 0.922 |
| Threonine | 3.23 | 3.45 | 0.935 |
| Serine | 4.32 | 4.49 | 0.962 |
| Glutamic Acid | 21.55 | 19.34 | 1.114 |
| Proline | 9.17 | 8.07 | 1.137 |
| Glycine | 2.57 | 3.43 | 0.749↓ |
| Alanine | 8.30 | 7.76 | 1.070 |
| Cysteine | 1.98 | 1.88 | 1.054 |
| Valine | 4.32 | 4.92 | 0.878 |
| Methionine | 3.15 | 3.53 | 0.892 |
| Isoleucine | 4.06 | 4.09 | 0.991 |
| Leucine | 15.12 | 12.88 | 1.174 |
| Tyrosine | 4.34 | 4.63 | 0.938 |
| Phenylalanine | 5.62 | 5.05 | 1.113 |
| Lysine | 1.38 | 2.92 | 0.472↓ |
| Histidine | 1.98 | 2.31 | 0.859 |
| Arginine | 2.23 | 3.87 | 0.576↓ |
| Tryptophan | 0.51 | 0.69 | 0.735↓ |

Table 3 shows that the soluble fractions from zein-enriched and zein-depleted have different amino acid compositions. The zein-enriched soluble fraction is depleted in arginine, glycine, lysine, and tryptophan compared to the zein-depleted fraction.

### Example 5—Amino Acid Composition Analysis of Water-Insoluble Refined Zein-Enriched and Zein-Depleted Protein Hydrolysate Compositions

The water-insoluble fractions of the refined zein-enriched protein hydrolysate composition and the refined zein-depleted protein hydrolysate composition prepared in Example 3 were evaluated to determine the respective amino acids content.

A comparison of the total amino acid content of water-insoluble zein-enriched (ZE-T) and zein-depleted (ZD-T) compositions expressed on a g/100 g protein basis is presented in Table 3, below. The ratio of enriched-to-depleted concentration is computed and ratios that differ by more than 20% are highlighted, along with an indicator of the deviation.

TABLE 4

| | ZE-I | ZD-I | |
|---|---|---|---|
| | g/100 g | g/100 g | |
| Amino acid | protein | protein | Enriched/Depleted |
| Aspartic Acid | 5.17 | 5.768256 | 0.897 |
| Threonine | 2.74 | 3.701826 | 0.739↓ |
| Serine | 4.53 | 3.904665 | 1.160 |
| Glutamic Acid | 23.52 | 17.98935 | 1.307↑ |
| Proline | 9.34 | 8.658722 | 1.078 |
| Glycine | 1.29 | 4.272312 | 0.302↓ |
| Alanine | 9.25 | 7.061359 | 1.310↑ |
| Cysteine | 0.93 | 3.004564 | 0.311↓ |
| Valine | 3.82 | 5.349899 | 0.713↓ |
| Methionine | 1.48 | 3.080629 | 0.482↓ |
| Isoleucine | 4.16 | 3.765213 | 1.106 |
| Leucine | 18.86 | 11.76471 | 1.603↑ |
| Tyrosine | 4.76 | 5.096349 | 0.934 |
| Phenylalanine | 6.75 | 5.261156 | 1.284↑ |
| Lysine | 0.16 | 2.877789 | 0.057↓ |
| Histidine | 1.34 | 2.814402 | 0.476↓ |
| Arginine | 1.64 | 4.563895 | 0.359↓ |
| Tryptophan | 0.25 | 1.064909 | 0.235↓ |

Table 4 shows that the water-insoluble fractions exhibit even greater differences in amino acid composition, with deviations for relative amounts of amino acids being different in both directions.

### Example 6—Amino Acid Composition Analysis of Water-Soluble and Water-Insoluble Refined Zein-Enriched Protein Hydrolysate Compositions

The water-soluble and water-insoluble fractions of the refined zein-enriched protein hydrolysate compositions prepared in Example 3 were evaluated to determine the respective amino acids content.

A comparison of the total amino acid content of the water-soluble and water-insoluble fractions within the zein-enriched fraction expressed on a g/100 g protein basis is presented in Table 5, below. The ratio of enriched-to-depleted concentration is computed and ratios that differ by more than 20% are highlighted, along with an indicator of the deviation.

TABLE 5

| | |
|---|---|
| Soluble | Insoluble |

| | | |
|---|---|---|
| Amino Acid | g/100 g protein | Soluble/Insoluble |

| | | | |
|---|---|---|---|
| Aspartic Acid | 6.17 | 5.17 | 1.193 |
| Threonine | 3.23 | 2.74 | 1.179 |
| Serine | 4.32 | 4.53 | 0.954 |
| Glutamic Acid | 21.55 | 23.52 | 0.916 |
| Proline | 9.17 | 9.34 | 0.982 |
| Glycine | 2.57 | 1.29 | 1.988↑ |
| Alanine | 8.30 | 9.25 | 0.898 |
| Cysteine | 1.98 | 0.93 | 2.120↑ |
| Valine | 4.32 | 3.82 | 1.133 |
| Methionine | 3.15 | 1.48 | 2.124↑ |
| Isoleucine | 4.06 | 4.16 | 0.975 |
| Leucine | 15.12 | 18.86 | 0.802 |
| Tyrosine | 4.34 | 4.76 | 0.912 |
| Phenylalanine | 5.62 | 6.75 | 0.833 |
| Lysine | 1.38 | 0.16 | 8.410↑ |
| Histidine | 1.98 | 1.34 | 1.479↑ |
| Arginine | 2.23 | 1.64 | 1.359↑ |
| Tryptophan | 0.51 | 0.25 | 2.034↑ |

Table 5 shows that the two fractions are unique from each other, particularly with respect to lysine concentrations.

### Example 7—Amino Acid Composition Analysis of Water-Soluble and Water-Insoluble Refined Zein-Depleted Protein Hydrolysate Compositions

The water-soluble and water-insoluble fractions of the refined zein-depleted protein hydrolysate compositions prepared in Example 3 were evaluated to determine the respective amino acids content.

A comparison of the total amino acid content of the water-soluble and water-insoluble fractions within the zein-enriched fraction expressed on a g/100 g protein basis is presented in Table 6, below. The ratio of enriched-to-depleted concentration is computed and ratios that differ by more than 20% are highlighted, along with an indicator of the deviation.

TABLE 6

| | |
|---|---|
| Soluble | Insoluble |

| | | |
|---|---|---|
| Amino Acid | g/g protein | Soluble/Insoluble |

| | | | |
|---|---|---|---|
| Aspartic Acid | 6.69 | 5.77 | 1.160 |
| Threonine | 3.45 | 3.70 | 0.933 |
| Serine | 4.49 | 3.90 | 1.151 |
| Glutamic Acid | 19.34 | 17.99 | 1.075 |
| Proline | 8.07 | 8.66 | 0.931 |
| Glycine | 3.43 | 4.27 | 0.802 |
| Alanine | 7.76 | 7.06 | 1.099 |
| Cysteine | 1.88 | 3.00 | 0.626↓ |
| Valine | 4.92 | 5.35 | 0.920 |
| Methionine | 3.53 | 3.08 | 1.147 |
| Isoleucine | 4.09 | 3.77 | 1.087 |
| Leucine | 12.88 | 11.76 | 1.095 |
| Tyrosine | 4.63 | 5.10 | 0.908 |
| Phenylalanine | 5.05 | 5.26 | 0.960 |
| Lysine | 2.92 | 2.88 | 1.015 |
| Histidine | 2.31 | 2.81 | 0.819 |
| Arginine | 3.87 | 4.56 | 0.847 |
| Tryptophan | 0.69 | 1.06 | 0.651↓ |

Table 6 shows that there is much less difference between soluble and insoluble fractions in this case, with the exception of the amino acids cysteine and tryptophan.

Examples 1-7 show that the amino acid composition profiles of each of these compositions is different from each other, so that these compositions would provide nutritional benefits that are different from each other, and additionally would have different optimal product uses. Because the amino acid profile of each of the different protein composition products is different from the others, the different protein products as described herein may advantageously be used in unique blends with supplements or other food components to provide a desired amino acid profile.

### Example 8

ZE and ZD Corn Protein compositions were prepared as described in Example 1A and B, and enzymatic hydrolysis was carried out to prepare ZE and ZD hydrolysate compositions as described in Example 1C, under the conditions of pH, temperature, enzyme to substrate ratio (E/S) and time of the hydrolysis as presented in Table 7.

Samples 8-1 through 8-12 and 8-14 to 8-16 were prepared once and the below reported properties were measured on a single sample, with the protein content solubility and DH measurements being taken twice and averaged. Sample 8-13 was prepared twice, and the properties measured twice on both samples, with the protein content, solubility and DH measurements being averaged. Samples 8-17 through 8-20 were prepared three times, and the properties measured twice on all three samples, with the protein content, solubility and DH measurements being averaged.

TABLE 7
Protein content, solubility and DH of ZE and ZD hydrolysates produced under different hydrolysis conditions.

| | | | | | ZE | | | | ZD | | | |
|---|---|---|---|---|---|---|---|---|---|---|---|---|
| | | | | | Protein | Solubility | Solubility | | Protein | Solubility | Solubility | |
| Sample | pH | Temp | E/S | Time | content | at pH 3.4 | at pH 7 | DH | content | at pH 3.4 | at pH 7 | DH |
| 8-1 | 5 | 45 | 1:50 | 120 | 85.17 | 17.22 | 25.53 | 9.80 | 75.16 | 37.98 | 38.38 | 13.20 |
| 8-2 | 5 | 45 | 1:25 | 60 | 86.13 | 19.00 | 19.80 | 8.50 | 71.77 | 34.36 | 35.57 | 13.11 |
| 8-3 | 5 | 50 | 01:37.5 | 90 | 84.23 | 26.33 | 26.45 | 10.28 | 72.63 | 40.34 | 42.72 | 14.62 |
| 8-4 | 5 | 55 | 1:50 | 120 | 85.83 | 23.88 | 25.06 | 11.32 | 72.86 | 37.12 | 39.25 | 16.29 |
| 8-5 | 5 | 55 | 1:25 | 60 | 85.17 | 24.74 | 23.69 | 12.04 | 74.26 | 39.02 | 39.14 | 16.02 |
| 8-6 | 5.5 | 45 | 1:50 | 90 | 86.14 | 19.69 | 20.33 | 6.85 | 74.85 | 27.17 | 27.99 | 11.12 |
| 8-7 | 5.5 | 50 | 1:25 | 120 | 85.99 | 28.41 | 29.34 | 15.36 | 72.36 | 51.71 | 52.52 | 23.64 |
| 8-8 | 6 | 45 | 01:37.5 | 60 | 88.34 | 10.16 | 9.74 | 2.69 | 74.62 | 21.74 | 20.60 | 8.05 |
| 8-9 | 6 | 45 | 1:25 | 120 | 85.68 | 19.94 | 20.41 | 10.78 | 71.78 | 43.04 | 45.07 | 19.47 |
| 8-10 | 6 | 50 | 1:50 | 60 | 86.13 | 12.31 | 9.82 | 1.34 | 73.91 | 22.85 | 23.19 | 6.84 |
| 8-11 | 6 | 55 | 01:37.5 | 120 | 87.65 | 16.72 | 18.26 | 6.71 | 72.53 | 33.87 | 40.77 | 16.29 |
| 8-12 | 6 | 55 | 1:25 | 90 | 88.98 | 19.62 | 20.28 | 4.31 | 75.21 | 40.40 | 40.76 | 14.23 |
| 8-13 | 5.5 | 50 | 1:50 | 60 | 90.68 | 16.99 | 16.23 | 4.16 | 76.05 | 26.37 | 28.27 | 7.80 |
| 8-14 | 5.5 | 60 | 1:50 | 60 | 88.95 | 8.70 | 7.80 | −0.12 | 74.42 | 27.35 | 27.55 | 7.68 |
| 8-15 | 6 | 60 | 1:25 | 120 | 85.81 | 13.29 | 12.65 | 4.69 | 72.96 | 38.62 | 37.84 | 14.99 |
| 8-16 | 5 | 60 | 1:50 | 60 | 88.58 | 13.32 | 13.47 | 2.83 | 76.61 | 26.18 | 27.77 | 6.95 |
| 8-17 | 5 | 55 | 1:50 | 72 | 89.86 | 18.75 | 18.04 | 5.73 | — | — | — | — |
| 8-18 | 5.1 | 54 | 1:25.6 | 120 | 86.95 | 32.36 | 31.77 | 17.34 | — | — | — | — |
| 8-19 | 6 | 49 | 1:38.5 | 60 | — | — | — | — | 76.02 | 24.52 | 24.12 | 7.18 |
| 8-20 | 5.1 | 51 | 1:25.6 | 120 | — | — | — | — | 73.58 | 51.27 | 52.0 | 18.03 |

### Example 9—Surface Hydrophobicity

Changes in the configuration of a protein may change the exposure of polar and non-polar amino acid side chains. The relative effect of such changes can be assessed by measuring the binding of a hydrophobic compound to the protein, a measure called surface hydrophobicity.

This spectrofluorimetric method uses an aromatic fluorescent probe, 1-aniline 8-napthalene sulfonate (ANS), which emits detectable light when excited by light of an appropriate wavelength (Kato and Nakai, 1980; Alizadeh-Pasdar and Li-Chan, 2000). An ANS stock solution was made by suspending 0.03976 g ANS in 10 mL 0.1M pH 7.4 phosphate buffer and storing the stock in the dark (stable for 6 months). A working solution of ANS was made fresh every working day by diluting the ANS stock solution of 133 μL in 3734 μL citric acid: sodium phosphate pH 7 buffer. Protein solutions were prepared (0.05% w/v) by weighing out the amount of powder necessary to reach 5 mg of protein each into 15 mL centrifuge tubes, adding 10 mL of 0.1M Phosphate buffer, pH 7.4 to each tube, and adjusting the pH to 7.0. Using the 0.05% protein solutions, concentrations of 0.025, 0.02, 0.015, 0.01 and 0.005 w/v were prepared. 200 μL of 0.005-0.050% protein samples were loaded into a black opaque 96 well plate. Blanks contained only citric acid:sodium phosphate pH buffer. All the samples and blanks were prepared in duplicate. The relative fluorescence index (RFI) was measured by setting the excitation and emission wavelengths at 400/30 (excitation wavelength/full width at half-maximum) and 460/40 nm, respectively. Gain was set to 25. 20 μL of ANS probe solution was added to each sample and blank. The plate was shaken for 1 min, then left sitting for 15 minutes in the dark before measuring the RFI again.

Calculation of net RFI: Blanks (wells containing only citric acid:sodium phosphate buffer or containing citric acid:sodium phosphate buffer with ANS added) in each plate were averaged separately. For each plate, appropriate blank average was subtracted from each sample. The net RFI was calculated by subtracting the RFI of the sample without added ANS probe from the RFI of the corresponding sample with ANS. Net RFI vs. protein concentration (%) was plotted a linear regression trend-line. The slope of the linear regression is the protein surface hydrophobicity.

Table 8 shows surface hydrophobicity of corn protein, ZE, ZD intact fractions, hydrolysates and their respective water soluble and insoluble fractions as identified in Table 7.

TABLE 8

| Sample | Average | Std. Dev. |
|---|---|---|
| Base CPI | 54723 | 5141 |
| Commercial Zein | 1610 | 510 |
| Intact ZE protein | 2050 | 90 |
| ZE 8-13 | 1776 | 91 |
| ZE_8-17 | 9842 | 543 |
| ZE_8-18 | 10677 | 743 |
| ZE 8-13_Soluble | 2703 | 49 |
| ZE_8-17_Soluble | 2833 | 40 |
| ZE_8-18_Soluble | 1812 | 129 |
| ZE 8-13_Insoluble | 17057 | 1027 |
| ZE_8-17_Insoluble | 14316 | 1804 |
| ZE_8-18_Insoluble | 18208 | 1881 |
| Intact ZD protein | 2742 | 39 |
| ZD 8-13 | 1813 | 92 |
| ZD_8-19 | 4058 | 184 |
| ZD_8-20 | 4735 | 515 |
| ZD 8-13_Soluble | 2502 | 77 |
| ZD_8-19_Soluble | 1919 | 22 |
| ZD_8-20_Soluble | 1856 | 187 |
| ZD 8-13_Insoluble | 13336 | 1071 |
| ZD_8-19_Insoluble | 6919 | 272 |
| ZD_8-20_Insoluble | 5664 | 556 |
| Whey protein isolate | 2470 | 84 |

Ethanol mediated fractionation of corn protein into ZE and ZD significantly reduced the surface hydrophobicity. Intact ZE and ZD were similar but surface hydrophobicity of ZE increased to a greater extent upon hydrolysis. Water mediated fractionation clearly resulted in a more hydrophobic insoluble portion and less hydrophobic soluble portion for both ZE and ZD.

### Example 10—Separation of Refined Zein-Enriched and Zein-Depleted Protein Hydrolysate Compositions into Water-Soluble and Water-Insoluble Compositions

The refined zein-enriched protein hydrolysate composition as prepared in Example 8 was separated into a water-soluble fraction and a water-insoluble fraction by treatment with water. Specifically, the water-soluble refined zein-enriched protein hydrolysate composition is extracted from the refined zein-enriched protein hydrolysate composition in the supernatant, leaving behind a water-insoluble refined zein-enriched protein hydrolysate composition as the extracted solids (residue). 5% (on protein basis) of zein-enriched protein hydrolysate was suspended in water, followed by pH adjustment to 7 and mixing for 1 hour at room temperature. The separation of the supernatant from the extracted solids is carried out by centrifugation. Both the water-soluble fraction and the water-insoluble fraction are dried by freeze-drying for subsequent analysis.

Likewise, the refined zein-depleted protein hydrolysate composition as prepared in Example 8 was separated into a water-soluble fraction and a water-insoluble fraction by treatment with water. Specifically, the water-soluble refined zein-depleted protein hydrolysate composition is extracted from the refined zein-depleted protein hydrolysate composition in the supernatant, leaving behind a water-insoluble refined zein-depleted protein hydrolysate composition as the extracted solids (residue). 5% (on protein basis) of zein-depleted protein hydrolysate was suspended in water, followed by pH adjustment to 7 and mixing for 1 hour at room temperature. The separation of the supernatant from the extracted solids is carried out by centrifugation. Both the water-soluble fraction and the water-insoluble fraction are dried by freeze-drying for subsequent analysis.

### Example 11—Amino Acid Composition of Water-Soluble and Water-Insoluble Fractions of Sample ZE 8-17

A comparison of the total amino acid content of the water-soluble and water-insoluble fractions of sample ZE 8-17 (as identified in Table 7) expressed on a g/100 g protein basis is presented in Table 9 below. The ratio of soluble-to-insoluble concentration is calculated and ratios that differ by more than 20% are marked with an indicator of the deviation.

TABLE 9

| | |
|---|---|
| Soluble | Insoluble |

| | | |
|---|---|---|
| Amino Acid | g/100 g protein | Soluble/Insoluble |

| | | | |
|---|---|---|---|
| Aspartic Acid | 5.67 | 5.16 | 1.10 |
| Threonine | 2.90 | 2.71 | 1.07 |
| Serine | 4.18 | 4.49 | 0.93 |
| Glutamic Acid | 22.79 | 23.61 | 0.97 |
| Proline | 9.11 | 9.02 | 1.01 |
| Glycine | 1.71 | 1.20 | 1.43↑ |
| Alanine | 9.09 | 9.32 | 0.98 |
| Cysteine | 1.24 | 0.87 | 1.43↑ |
| Valine | 4.07 | 3.76 | 1.08 |
| Methionine | 1.81 | 1.40 | 1.29↑ |
| Isoleucine | 4.47 | 4.28 | 1.04 |
| Leucine | 17.72 | 19.31 | 0.92 |
| Tyrosine | 4.61 | 4.80 | 0.96 |
| Phenylalanine | 6.46 | 6.91 | 0.93 |
| Lysine | 0.56 | 0.09 | 6.34↑ |
| Histidine | 1.50 | 1.28 | 1.17 |
| Arginine | 1.76 | 1.57 | 1.13 |
| Tryptophan | 0.35 | 0.23 | 1.49↑ |

Table 9 shows that the two fractions are comparable to each other, except for higher concentration of lysine, glycine, cysteine and tryptophan in the water soluble fraction compared to that of the water insoluble fraction.

### Example 12—Amino Acid Composition of Water-Soluble and Water-Insoluble Fractions of Sample ZE 8-18

A comparison of the total amino acid content of the water-soluble and water-insoluble fractions of sample, ZE 8-18 (as identified in Table 7) expressed on a g/100 g protein basis is presented in Table 10. The ratio of soluble-to-insoluble concentration is calculated and ratios that differ by more than 20% are marked with an indicator of the deviation.

TABLE 10

| | |
|---|---|
| Soluble | Insoluble |

| | | |
|---|---|---|
| Amino Acid | g/100 g protein | Soluble/Insoluble |

| | | | |
|---|---|---|---|
| Aspartic Acid | 5.64 | 5.17 | 1.09 |
| Threonine | 2.91 | 2.69 | 1.08 |
| Serine | 4.52 | 4.23 | 1.07 |
| Glutamic Acid | 22.85 | 23.67 | 0.97 |
| Proline | 9.07 | 9.20 | 0.99 |
| Glycine | 1.59 | 1.21 | 1.31↑ |
| Alanine | 9.13 | 9.35 | 0.98 |
| Cysteine | 1.11 | 0.85 | 1.30↑ |
| Valine | 3.99 | 3.81 | 1.05 |
| Methionine | 1.60 | 1.39 | 1.15 |
| Isoleucine | 4.51 | 4.39 | 1.03 |
| Leucine | 17.95 | 19.27 | 0.93 |
| Tyrosine | 4.64 | 4.65 | 1.00 |
| Phenylalanine | 6.55 | 6.95 | 0.94 |
| Lysine | 0.48 | 0.09 | 5.47↑ |
| Histidine | 1.42 | 1.29 | 1.10 |
| Arginine | 1.68 | 1.56 | 1.07 |
| Tryptophan | 0.37 | 0.23 | 1.61↑ |

Table 10 shows that the two fractions are comparable to each other except for higher concentration of lysine, glycine, cysteine and tryptophan in the water soluble fraction compared to that of the water insoluble fraction.

### Example 13—Amino Acid Composition of Water-Soluble and Water-Insoluble Fractions of Sample ZD 8-19

A comparison of the total amino acid content of the water-soluble and water-insoluble fractions of sample ZD 8-19 (as identified in Table 7), expressed on a g/100 g protein basis is presented in Table 11. The ratio of soluble-to-insoluble concentration is calculated and ratios that differ by more than 20% are marked with an indicator of the deviation.

TABLE 11

| | |
|---|---|
| Soluble | Insoluble |

| | | |
|---|---|---|
| Amino Acid | g/100 g protein | Soluble/Insoluble |

| | | | |
|---|---|---|---|
| Aspartic Acid | 7.20 | 5.60 | 1.29↑ |
| Threonine | 3.57 | 3.65 | 0.98 |
| Serine | 4.09 | 3.89 | 1.05 |
| Glutamic Acid | 19.09 | 18.14 | 1.05 |
| Proline | 7.55 | 8.80 | 0.86 |
| Glycine | 3.56 | 4.24 | 0.84 |
| Alanine | 7.88 | 6.97 | 1.13 |
| Cysteine | 1.79 | 3.17 | 0.56↓ |
| Valine | 5.17 | 5.42 | 0.95 |
| Methionine | 3.89 | 2.96 | 1.31↑ |
| Isoleucine | 4.48 | 3.91 | 1.15 |
| Leucine | 12.67 | 11.76 | 1.08 |
| Tyrosine | 4.14 | 4.94 | 0.84 |
| Phenylalanine | 4.85 | 5.35 | 0.91 |
| Lysine | 3.22 | 2.78 | 1.16 |
| Histidine | 2.19 | 2.88 | 0.76↓ |
| Arginine | 3.93 | 4.54 | 0.87 |
| Tryptophan | 0.75 | 1.00 | 0.75↓ |

Table 11 shows that the two fractions are unique from each other, particularly with respect to aspartic acid, methionine and cysteine concentrations.

### Example 14—Amino Acid Composition of Water-Soluble and Water-Insoluble Fractions of Sample ZD 8-20

A comparison of the total amino acid content of the water-soluble and water-insoluble fractions of sample ZD 8-20 (as identified in Table 7), expressed on a g/100 g protein basis is presented in Table 12. The ratio of soluble-to-insoluble concentration is calculated and ratios that differ by more than 20% are marked with an indicator of the deviation.

TABLE 12

| | |
|---|---|
| Soluble | Insoluble |

| | | |
|---|---|---|
| Amino Acid | g/100 g protein | Soluble/Insoluble |

| | | | |
|---|---|---|---|
| Aspartic Acid | 6.82 | 5.28 | 1.29↑ |
| Threonine | 3.58 | 3.81 | 0.94 |
| Serine | 4.05 | 3.90 | 1.04 |
| Glutamic Acid | 18.28 | 18.08 | 1.01 |
| Proline | 7.99 | 9.00 | 0.89 |
| Glycine | 3.66 | 4.70 | 0.78↓ |
| Alanine | 7.48 | 6.78 | 1.10 |
| Cysteine | 2.11 | 3.65 | 0.58↓ |
| Valine | 5.31 | 5.44 | 0.98 |
| Methionine | 3.38 | 2.85 | 1.19 |
| Isoleucine | 4.37 | 3.70 | 1.18 |
| Leucine | 12.34 | 11.06 | 1.12 |
| Tyrosine | 4.63 | 5.00 | 0.93 |
| Phenylalanine | 5.12 | 5.38 | 0.95 |
| Lysine | 3.22 | 2.71 | 1.19 |
| Histidine | 2.49 | 2.98 | 0.83 |
| Arginine | 4.32 | 4.58 | 0.94 |
| Tryptophan | 0.85 | 1.09 | 0.78↓ |

Table 12 shows that the two fractions are unique from each other, particularly with respect to aspartic acid and cysteine concentrations.

### Example 15—Protein Secondary Structure by Attenuated Total Reflectance-Fourier Transform Infrared Spectroscopy (ATR-FTIR)

ZE and ZD samples were analyzed by Thermo Scientific Nicolet iS10 FTIR spectrometer (Thermo Fisher Scientific, Waltham, Mass.) equipped with a horizontal multi reflectance diamond accessary using the OMNIC 8 software. Secondary structures of intact and hydrolyzed samples were determined from second-derivative spectra of amide I regions (1600-1700 cm⁻¹). Spectral regions were assigned as 1600-1635 for β-sheets, 1636-1649 cm⁻¹ for α-helix, 1650-1680 cm⁻¹ for random, and 1681-1700 cm⁻¹ for β-turn structures. The second derivative area for each secondary structural region was divided by the total area of the amide I region. A minimum of 3 spectra were recorded per sample. Results are reported in Table 13 below.

TABLE 13
Protein secondary structure of ZE and ZD hydrolysates

| | β sheet | α helix | random | β turn |
|---|---|---|---|---|
| CPI | 15.85 | 51.64 | 25.39 | 7.12 |
| Commercial Zein | 3.64 | 64.44 | 24.97 | 6.95 |
| Intact ZE protein | 8.47 | 58.53 | 24.80 | 8.21 |
| Intact ZD protein | 12.62 | 52.06 | 26.26 | 9.07 |
| ZE 8-13 | 7.38 | 54.99 | 29.28 | 8.35 |
| ZE 8-13 Soluble | 34.65 | 26.61 | 28.51 | 10.23 |
| ZE 8-13 Insoluble | 37.90 | 30.60 | 19.45 | 12.05 |
| ZE 8-17 | 6.12 | 49.51 | 36.74 | 7.63 |
| ZE 8-17 Soluble | 33.56 | 24.70 | 30.16 | 11.58 |
| ZE 8-17 Insoluble | 40.56 | 28.57 | 19.94 | 10.93 |
| ZE 8-18 | 4.13 | 45.62 | 42.5 | 7.76 |
| ZE 8-18 Soluble | 34.19 | 22.48 | 31.39 | 11.93 |
| ZE 8-18 Insoluble | 40.43 | 28.24 | 20.37 | 10.96 |
| ZD 8-13 | 9.55 | 50.19 | 30.86 | 9.36 |
| ZD 8-13 Soluble | 33.14 | 28.14 | 26.07 | 12.65 |
| ZD 8-13 Insoluble | 38.11 | 27.14 | 18.37 | 16.38 |
| ZD 8-19 | 5.98 | 48 | 36.06 | 9.95 |
| ZD 8-19 Soluble | 32.65 | 28.10 | 26.24 | 13.01 |
| ZD 8-19 Insoluble | 39.89 | 26.51 | 18.78 | 14.82 |
| ZD 8-20 | <0.02 | 43.46 | 47.1 | 9.64 |
| ZD 8-20 Soluble | 33.56 | 26.12 | 27.11 | 13.21 |
| ZD 8-20 Insoluble | 39.21 | 26.42 | 18.53 | 15.83 |

This data shows that compositions hydrolyzed under different conditions exhibit different major protein secondary structures.

### Example 16—the Effect of Thermal Treatment on Protein Solubility

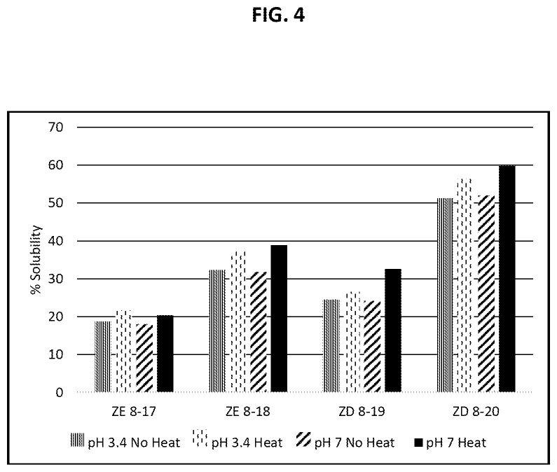

Solubility of the samples ZE 8-17, ZE 8-18, ZD 8-19 and ZD 8-20 (as identified in Table 7) were determined at pH 3.4 and 7 with and without thermal treatment (85° C. for 30 minutes) under the method described in Example 1D. Solubility results are shown in FIG. 4. It was found that thermal treatment enhanced the solubility of all the samples regardless of the pH tested. The evaluation confirmed that thermal treatment did not these samples did not induce protein aggregation or reduced solubility. Thermal stability of the present compositions is particularly beneficial for use in product formats that may be exposed to heat, such as in beverage applications.

### Example 17—the Effect of Suspension pH on Protein Solubility

Solubility of the hydrolyzed ZE and ZD were determined at pH 3, 4, 5, 6, 7, 8 and 9 as mentioned in Example 1D.

One sample of each of the formulations 8-13 and 8-17 to 8-20 was selected for measurement of protein solubility. One solution of each of the formulations was prepared at each indicated pH level, and the protein solubility was measured twice and averaged. Proteins are heterogeneous polymers comprising potentially positively, negatively, and neutral amino acids side chains. Solubility is enhanced when the protein has a net charge and may be minimized when the net charge on the protein is nearly zero. Most proteins precipitate near their isoelectric point, which is often between 4 and 6 (ex: whey protein isolate and casein).

The effect of suspension pH on solubility of samples ZE 8-13, ZE 8-17, ZE 8-18, ZD 8-13, ZD 8-19 and ZD 8-20 (as identified in Table 7) are shown in Table 14.

TABLE 14

| | pH | pH | pH | pH | pH | pH | pH |
|---|---|---|---|---|---|---|---|
| | 3.0 | 4.0 | 5.0 | 6.0 | 7.0 | 8.0 | 9.0 |
| ZE 8-13 | 17.92 | 17.93 | 18.15 | 17.46 | 15.28 | 14.73 | 21.20 |
| ZE 8-17 | 12.71 | 15.56 | 17.27 | 16.72 | 15.08 | 16.05 | 16.41 |
| ZE 8-18 | 35.61 | 35.52 | 34.93 | 35.47 | 37.48 | 37.34 | 36.83 |
| ZD 8-13 | 28.70 | 26.95 | 30.06 | 28.83 | 26.55 | 31.50 | 31.31 |
| ZD 8-19 | 20.10 | 19.69 | 21.65 | 21.34 | 22.80 | 25.21 | 25.61 |
| ZD 8-20 | 46.99 | 45.96 | 50.95 | 49.73 | 51.68 | 50.01 | 50.67 |

ZD samples behave differently from that of ZE. The ZD 8-19 and ZD 8-20 show higher solubility above pH 7 compared to that of below pH 7. The solubility of all ZE samples are insensitive to changes in the suspension Ph.

The protein stability of solubility in acidic conditions is desirable in many foods processes where pH modification occurs.

### Example 18—Bitterness Evaluation

Samples for sensory evaluation were prepared by dispersing corn protein (5% w/w) and bitter reference standard caffeine (#1=0.107 g/Kg, #2=0.153 g/Kg, #3=0.2 g/Kg, #4=0.246 g/Kg and #5=0.293 g/Kg) into deionized water and stored overnight at 40° F. Protein solutions (100 ml) were passed through a Nalgene Rapid Flow filter unit (1000 ml capacity, 90 mm diameter, 0.2 μm pore, PES membrane) prior to serving. A total of 20 individuals familiarized themselves with the series of caffeine reference solutions prior to evaluating the protein samples. Data collected from panelists (3) who were not able to distinguish differences among the caffeine standard solutions were removed from subsequent analysis. To taste protein solution samples, evaluators dispensed approximately 3.5 mL of each solution into their own mouths and dispersed by moving their tongues followed by spiting the sample. After spitting, panelists assigned a bitterness score compared to their perception of the caffeine reference solutions. Between samples, panelists had ad libitum access to water and rice crackers for palate cleansing. Mean scores and confidence intervals were calculated from the data and are listed in Table 15. Some panelists found the bitter sensation to be less than 1 or greater than 5; these values were assigned 0 and 6, respectively for analysis.

TABLE 15

| | Average | 95% CI |
|---|---|---|
| Treatment | Bitter Score | (+/−) |
| Intact ZD protein | 1.62 | 0.57 |
| ZD 8-13 | 2.69 | 0.54 |
| ZD 8-19 | 2.90 | 0.66 |
| ZD 8-20 | 4.91 | 0.49 |
| Intact ZE protein | 2.69 | 0.86 |
| ZE 8-13 | 3.93 | 0.67 |
| ZE 8-17 | 3.69 | 0.68 |
| ZE 8-18 | 4.22 | 0.89 |

The bitterness evaluation demonstrated that compositions as described herein can be adapted to present very different flavor profiles, and therefore can be tailored for use in different applications based on selection of hydrolysis conditions and the resulting fractions prepared.

All patents, patent applications (including provisional applications), and publications cited herein are incorporated by reference as if individually incorporated for all purposes. Unless otherwise indicated, all parts and percentages are by weight and all molecular weights are weight average molecular weights. The above detailed description includes references to the accompanying drawings, which form a part of the detailed description. The drawings show, by way of illustration, specific aspects in which the invention can be practiced. These aspects are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

1. A method of preparing a refined zein-enriched protein hydrolysate composition and/or a refined zein-depleted protein hydrolysate composition, comprising:
a) providing a refined zein-enriched protein composition and/or a refined zein-depleted protein composition,
wherein the refined zein-enriched protein composition and/or the refined zein-depleted protein composition have been separated from a refined, destarched corn gluten meal that
i) comprises less than about 2 wt % oil on a dry basis,
ii) has an L* color value of from about 88 to 95, an “a*” color value ranging from about −0.5 to 1.5, and a “b*” color value ranging from about 10 to 25, iii) has a soluble carbohydrate concentration of 40 g/kg or less, and iv) comprises at least about 85 wt % protein on a dry basis;
the refined, destarched corn gluten meal having been prepared by treating a destarched corn gluten meal with a water-miscible solvent/water extraction composition comprising from about 55% to about 90% water-miscible solvent;
b) adding an enzyme to the refined zein-enriched protein composition to provide a zein-enriched protein suspension, and/or
adding an enzyme to the refined zein-depleted protein composition to provide a zein-depleted protein suspension;
c) controlling the pH and temperature of the zein-enriched protein suspension and/or the zein-depleted protein suspension to hydrolyze protein in the zein-enriched protein suspension and/or the zein-depleted protein suspension; and
d) terminating the hydrolysis of the zein-enriched protein suspension and/or the zein-depleted protein suspension to provide
i) a refined zein-enriched protein hydrolysate composition having a protein solubility of from about 15% to about 20% at a pH selected from the group consisting of pH 3.4, pH 7.0, and of both pH 3.4 and pH 7.0; and/or
ii) a refined zein-depleted protein hydrolysate composition having a protein solubility of from about 20% to about 35% at a pH selected from the group consisting of pH 3.4, pH 7.0, and of both pH 3.4 and pH 7.0.

2. A method of preparing a refined zein-enriched protein hydrolysate composition and/or a refined zein-depleted protein hydrolysate composition, comprising:
a) providing a refined zein-enriched protein composition and/or a refined zein-depleted protein composition,
wherein the refined zein-enriched protein composition and/or the refined zein-depleted protein composition have been separated from a corn protein isolate, the corn protein isolate having a corn protein concentration of at least 85% (ds), wherein from about 40% to about 70% of the corn protein is soluble in a 65% ethanol/water solution at a temperature of 60° C.;
b) adding an enzyme to the refined zein-enriched protein composition to provide a zein-enriched protein suspension, and/or
adding an enzyme to the refined zein-depleted protein composition to provide a zein-depleted protein suspension;
c) controlling the pH and temperature of the zein-enriched protein suspension and/or the zein-depleted protein suspension to hydrolyze protein in the zein-enriched protein suspension and/or the zein-depleted protein suspension; and
d) terminating the hydrolysis of the zein-enriched protein suspension and/or the zein-depleted protein suspension to provide
i) a refined zein-enriched protein hydrolysate composition having a protein solubility of from about 15% to about 20% at a pH selected from the group consisting of pH 3.4, pH 7.0, and of both pH 3.4 and pH 7.0; and/or
ii) a refined zein-depleted protein hydrolysate composition having a protein solubility of from about 20% to about 35% at a pH selected from the group consisting of pH 3.4, pH 7.0, and of both pH 3.4 and pH 7.0.

3. The method of claim 1, wherein the refined zein-enriched protein hydrolysate composition has a protein solubility of from about 16% to about 20% at a pH selected from the group consisting of pH 3.4, pH 7.0, and of both pH 3.4 and pH 7.0.

4. The method of claim 1, wherein the refined zein-depleted protein hydrolysate composition has a protein solubility of from about 22% to about 32% at a pH selected from the group consisting of pH 3.4, pH 7.0, and of both pH 3.4 and pH 7.0.

5. The method of claim 1, wherein the refined zein-enriched protein hydrolysate composition has a degree of hydrolysis of from about 3% to about 5%.

6. The method of claim 1, wherein the refined zein-depleted protein hydrolysate composition has a degree of hydrolysis of from about 5.5% to about 9%; or wherein the refined zein-depleted protein hydrolysate composition has a degree of hydrolysis of from about 5.5% to about 7%.

7. The method of claim 1, wherein the ratio of enzyme to protein is from about 1:100 to about 1:20 by weight.

8. (canceled)

9. The method of claim 1, wherein the pH of the protein suspension during hydrolysis is from about 5.0 to about 6.0.

10. The method of claim 1, wherein the temperature of the protein suspension during hydrolysis is from about 40° C. to about 60° C.

11. (canceled)

12. The method of claim 1, wherein the hydrolysis of the protein suspension is terminated by neutralizing the protein suspension to pH 7.0 and inactivating the enzyme by heating to a temperature of at least about 75° C. for at least about 5 minutes.

13. The method of claim 1, wherein the protein concentration of the refined zein-enriched protein hydrolysate composition and/or the refined zein-depleted protein hydrolysate composition is from about 75% to about 95%.

14. The method of claim 1, further comprising a step of separating the water-soluble proteins from the water-insoluble proteins in the refined zein-enriched protein hydrolysate composition to provide a water-soluble refined zein-enriched protein hydrolysate composition and a water-insoluble refined zein-enriched protein hydrolysate composition.

15. The method of claim 1, further comprising a step of separating the water-soluble proteins from the water-insoluble proteins in the refined zein-depleted protein hydrolysate composition to provide a water-soluble refined zein-depleted protein hydrolysate composition and a water-insoluble refined zein-depleted protein hydrolysate composition.

16-47. (canceled)

48. The method of claim 2, wherein the refined zein-enriched protein hydrolysate composition has a protein solubility of from about 16% to about 20% at a pH selected from the group consisting of pH 3.4, pH 7.0, and of both pH 3.4 and pH 7.0.

49. The method of claim 2, wherein the refined zein-depleted protein hydrolysate composition has a protein solubility of from about 22% to about 32% at a pH selected from the group consisting of pH 3.4, pH 7.0, and of both pH 3.4 and pH 7.0.

50. The method of claim 2, wherein the refined zein-enriched protein hydrolysate composition has a degree of hydrolysis of from about 3% to about 5%.

51. The method of claim 2, wherein the refined zein-depleted protein hydrolysate composition has a degree of hydrolysis of from about 5.5% to about 9%; or wherein the refined zein-depleted protein hydrolysate composition has a degree of hydrolysis of from about 5.5% to about 7%.

52. The method of claim 2, wherein the ratio of enzyme to protein is from about 1:100 to about 1:20 by weight.

53. The method of claim 2, wherein the pH of the protein suspension during hydrolysis is from about 5.0 to about 6.0.

54. The method of claim 2, wherein the temperature of the protein suspension during hydrolysis is from about 40° C. to about 60° C.

55. The method of claim 2, wherein the hydrolysis of the protein suspension is terminated by neutralizing the protein suspension to pH 7.0 and inactivating the enzyme by heating to a temperature of at least about 75° C. for at least about 5 minutes.

56. The method of claim 2, wherein the protein concentration of the refined zein-enriched protein hydrolysate composition and/or the refined zein-depleted protein hydrolysate composition is from about 75% to about 95%.

57. The method of claim 2, further comprising a step of separating the water-soluble proteins from the water-insoluble proteins in the refined zein-enriched protein hydrolysate composition to provide a water-soluble refined zein-enriched protein hydrolysate composition and a water-insoluble refined zein-enriched protein hydrolysate composition.

58. The method of claim 2, further comprising a step of separating the water-soluble proteins from the water-insoluble proteins in the refined zein-depleted protein hydrolysate composition to provide a water-soluble refined zein-depleted protein hydrolysate composition and a water-insoluble refined zein-depleted protein hydrolysate composition.
